(19) United States
(12) Patent Application Publication
(10) Pub. No.: US 2007/0197495 A1
(43) Pub. Date: Aug. 23, 2007

(54) ANTI-PARASITIC COMPOUNDS AND METHODS OF THEIR USE

(75) Inventor: Kelly Chibale, CLAREMONT (ZA)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: THE REGENTS OF THE UNIVERSITY OF CALIFORNIA, OAKLAND, CA (US)

(21) Appl. No.: 10/590,465

(22) PCT Filed: Mar. 7, 2005
(86) PCT No.: PCT/US05/07297

§ 371 Date: Aug. 23, 2006

Related U.S. Application Data
(60) Provisional application No. 60/550,699, filed on Mar. 5, 2004.

(51) Int. Cl. C07F 17/02; A61K 31/555; A61K 31/295

(52) U.S. Cl. 514/184; 514/185; 546/2; 514/502; 556/32

(57) ABSTRACT

The present invention provides a novel class of compounds that disrupt the parasitic infectious life cycle and serve as promising agents for anti-parasitic therapy.

### CROSS-REFERENCES TO RELATED APPLICATIONS

The present application claims the benefit of U.S. Ser. No. 60/550,699, filed Mar. 5, 2004, herein incorporated by reference in its entirety.

### STATEMENT AS TO RIGHTS TO INVENTIONS MADE UNDER FEDERALLY SPONSORED RESEARCH OR DEVELOPMENT

This invention was made with Government support under Grant Nos. CA 09270-27, awarded by the National Institutes of Health. The Government has certain rights in this invention.

### BACKGROUND OF THE INVENTION

Thiosemicarbazones are a class of small molecules that have been evaluated over the last 50 years as antivirals (Mishra et al., Arch Pharm (Weinheim), 335, 183-186 (2002); Condit et al., Virology, 185, 857-861 (1991)), and as anticancer therapeutics (Finch et al., Adv Enzyme Regul, 39, 3-12 (1999)), as well as for their parasiticidal action against Plasmodium falciparum (Klayman et al., J. Med. Chem., 22, 855-862 (1979); Scovill et al., U.S. Pat. No. 4,440,771; Klayman et al., U.S. Pat. No. 4,739,069; Klayman et al., U.S. Pat. No. 4,385,055) and Trypanosoma cruzis (Wilson et al., J. Med. Chem., 17, 760-761 (1974); Du et al., J. Med. Chem., 45, 2695-2707 (2002)), which are the causative agents of falciparum malaria and Chagas' Disease, respectively. Currently, a thiosemicarbazone, Triapine, is being evaluated in human phase II trials as an antineoplastic therapeutic (Feun et al., Cancer Chemother Pharmacol, 50, 223-229 (2002)). Electrochemical studies have been performed on semicarbazones and thiosemicarbazones derived from ferrocene (Graudo et al., J. Braz. Chem. Soc., 11, 237-240 (2000)).

A series of thiosemicarbazones was recently shown to inhibit a Trypanosoma cruzi derived cysteine protease, cruzain (Du et al., J. Med. Chem., 45, 2695-2707 (2002)). This study generated basic structure activity relationships (SAR) against cruzain.

Trypanosomiasis, malaria, Leishmaniasis, and trichomoniasis are major parasitic diseases in developing countries (McKerrow, J. H. et al., Annu. Rev. Microbiol. 47:821-853 (1993)). For example, American trypanosomiasis, or Chagas' disease, is the leading cause of heart disease in Latin America (Libow, L. F. et al., Cutis, 48:37-40 (1991)). At least 16-18 million people are infected with Trypanosoma cruzi, resulting in more than 50,000 deaths each year (Godal, T. et al., J. Tropical diseases. WHO Division of Control in Tropical Diseases World Health Organization: Geneva, Switzerland, pp 12-13. (1990); World Health Organization website: http://www.who.int/ctd/html/chagburtre.html). The statistics for malaria are more sobering, with about 300-500 million clinical cases and about 3 million deaths each year. Further, at least 10 million people are infected with a form of Leishmania each year (see Goodman & Gilman's The Pharmacological Basis of Therapeutics, 9^{th} Ed, 1996, McGraw-Hill, New York).

Chagas' disease is transmitted to humans by blood-sucking triatomine vectors with an infectious trypomastigote form of the protozoan parasite T. Cruzi (Bonaldo, M. C. et al., Exp. Parasitol, 73:44-51 (1981); Harth, G., et al., T. Cruzi. Mol. Biochem. Parasitol, 58:17-24 (1993); Meirelles, M. N. L., et al., Mol. Biochem. Parasitol, 52:175-184 (1992)). African trypanosomiasis is transmitted to humans and cattle by tsetse flies and is caused by subspecies of T. brucei. So called “African sleeping sickness” is transmitted by an infectious trypomastigote from T. brucei gambiense, and T. brucei rhodesiense produces a progressive and usually fatal form of disease marked by early involvement of the central nervous system. T. brucei is further the cause of nagana in cattle, but bovine trypanosomiasis is also transmitted by T. congolense and T. evansi. In trypanosomiasis infections, the trypomastigote enters the host bloodstream and ultimately invades a cardiac muscle cell, where it transforms into the intracellular amastigote. The parasite may also be found in the blood, lymph, spinal fluid and cells of the gastrointestinal tract. Amastigotes replicate within cells, transform back to trypomastigotes, and rupture the cell, releasing the infectious form back into the bloodstream and other cells, amplifying the infection. Reviews of the current understanding and treatment of African and American trypanosomiasis infections is provided by Urbina (Curr Pharm Des (2002) 8:287) and Burchmore, et al. (Curr Pharm Des (2002) 8:256).

Cruzain (aka cruzipain) is the major cysteine protease of T. cruzi. The protease is expressed in all life cycle stages of the parasite, but delivered to different cellular compartments in each stage. In the epimastigote stage, which occurs in the insect vector, the protease is in a lysosomal compartment where it functions to degrade proteins endocytosed from the insect gut. In the infectious trypomastigote stage, the protease appears at the flagellar pocket, the site of endocytosis and secretion. In the amastigote stage, within the mammalian host cell, the protease is both in the lysosomal compartment and on the surface of the parasite where it may function in nutrition, remodeling of the mammalian cell, or evasion of host defense mechanisms. Addition of a cruzain inhibitor such as Z-Phe-Ala-FMK (benzyloxy-carbonyl-L-phenylalanyl L-alanine fluoromethyl ketone) to cultures of mammalian cells exposed to trypomastigotes or to mammalian cells already infected with T. cruzi amastigotes blocks replication and differentiation of the parasite (Bonaldo, M. C. et al., Exp. Parasitol, 73:44-51 (1981); Harth, G., et al., T. Cruzi. Mol. Biochem Parasitol, 58:17-24 (1993); Meirelles, M. N. L., et al., Mol. Biochem. Parasitol, 52:175-184 (1992)), thus arresting the parasite life cycle. Therefore, cruzain is essential for replication of the intracellular parasite. Treatment of T. cruzi-infected mice with a vinyl sulfone-derivatized pseudopeptide inhibitor of cruzain, N-methyl piperazine-Phe-homoPhe-vinyl sulfone phenyl, has resulted in a cure in a mouse model study (Engel, J. C. et al., J. Exp. Med., 188:725-734 (1998)). Thus, cruzain is an appealing target for new antiTrypanosomal chemotherapy (McKerrow, J. H. et al., Bioorg. Med. Chem., 7:639-644 (1999)).

Malaria is caused by protozoa of the genus Plasmodium and is transmitted to humans through the bite of an infected anopheline mosquito. The parasites develop into tissue schizonts in hepatic parenchymal cells, and then are released into the circulation as merozoites, which invade erythrocytes. In erythrocytes, the merozoites mature from trophozoites into schizonts. Schizont-containing erythrocytes rupture to release merozoites that then invade more erythrocytes to continue the malarial cycle. Current understanding and treatment of Plasmodium infections is reviewed in Winstanley (Lancet Infect Dis (2001) 1:206), Wongsrichanalai, et al. (Lancet Infect Dis (2002) 2:209) and throughout the Feb. 7, 2002 issue of Nature (Lond) (vol. 415, issue 6872).

The majority of malaria infections is caused by Plasmodium falciparum (see Goodman & Gilman's The Pharmacological Basis of Therapeutics, supra). There are several Papain-family cysteine proteases, and several are thought to be essential to Plasmodium trophozoite protein synthesis and development (Sijwali, et al (2001) Biochem J 360:481) (Greenbaum et al (2002) Science 298:2002-2006. Sequencing of the Plasmodium genome has revealed at least three falcipain cysteine proteases, designated falcipain-1, falcipain-2 and falcipain-3, where falcipain-2 is understood to account for the majority of hemoglobinase activity in the Plasmodium trophozoite (Joachimiak, et al (2001) Mol. Med 7:698). The falcipains are homologous to cruzain (Venturini, et al (2000) Biochem Biophys Res Commun 270:437 and Selzer, et al (1997) Exp Parasitol 87:212) and the falcipain-2 and falcipain 1 sequences are highly conserved amongst different Plasmodium strains with different sensitivities to established antimalarial drugs (Singh and Rosenthal (2001) Antimicrob Agents Chemother 45:949). In in vitro studies, cysteine protease inhibitors blocked globin hydrolysis in Plasmodium infected erythrocytes (Rosenthal (1995) Exp. Parasitol 80:272 and Semenov et al (1998) Antimicrob Agents Chemother 42:2254). Importantly, oral or parenteral administration of fluoromethyl ketone or vinyl sulfone peptidyl inhibitors of falcipain cured treated mice that were infected with Plasmodium (Olson, et al (1999) Bioorg Med Chem 7:633). Therefore, the falcipains and other homologous cysteine proteases are also important antimalarial chemotherapeutic targets.

Leishmaniasis is caused by protozoal species and subspecies of Leishmania transmitted to humans by the bites of infected female phlebotamine sandflies. Promastigotes injected into the host are phagocytized by tissue monocytes and are transformed into amastigotes, which reside in intracellular phagolysosomes. Human Leishmaniasis is classified into cutaneous, mucocutaneous and visceral (kala azar) forms. Reviews of the current understanding and chemotherapy of Leishmaniasis is provided by Croft and Yardley (Curr Pharm Des (2002) 8:319), Kafetzis, et al (Curr Opin Infect Dis (2002) 15:289, and Hepburn (Curr Opin Infect Dis 14:151).

In vitro and in vivo studies also have demonstrated that cysteine protease inhibitors disrupt the infectious life cycle of Leishmania (see, Selzer, et al (1999) Proc Natl Acad Sci 96:11015; Das, et al (2001) J. Immunol 166:4020 and Salvati, et al (2001) Biochim Biophys Acta 1545:357). Like Trypanosoma and Plasmodium, Leishmania synthesize cathepsin-L-like cysteine proteases that are essential to their pathogenicity (Selzer, et al (1997) Exp Parasitol 87:212). The substrate recognition of one cysteine protease of L. mexicana, named CPB2.8 Delta CTE, has been demonstrated to be similar to the substrate preferences of cruzain (Alves, et al (2001) Mol Biochem Parasitol 117:137 and Alves, et al (2001) Mol Biochem Parasitol 116:1). Additionally, cruzain shares sequence similarity with homologous cysteine proteases from L. pifanoi, L. mexicana, and L. major (see Mottram, et al (1992) Mol Microbiol 6:1925, Rafati, et al (2001) Mol Biochem Parasitol 113:35 and GenBank numbers L29168, X62163 and AJ130942). Therefore, cysteine proteases also represent a potential chemotherapeutic target against Leishmania infections.

Trichomoniasis is a common sexually transmitted disease (STD) that affects 2 to 3 million Americans yearly. Trichomoniasis is caused by the single-celled protozoan parasite, Trichomonas vaginalis. Trichomoniasis is primarily an infection of the urogenital tract. The urethra and prostate is the most common site of infection in men, and the vagina is the most common site of infection in women.

Drugs currently used in the treatment of trypanosomiasis include Nifurtimox, Benznidazole, Suramin, Pentamidine isethionate, Eflornithine and Melarsoprol. Current chemotherapeutics for the treatment of Leishmaniasis include Stibogluconate sodium, Amphotericin B, and Pentamidine isethionate. Drugs used in the treatment of malaria include chloroquine phosphate, mefloquine, halofantrine, and quinidine gluconate in combination with an antifolate or an antibiotic. Drugs for treatment of Trichomoniasis include oral metronidazole. Although these protozoans are inhibited to some extent by the administration of available chemotherapeutics, the currently prescribed pharmacological compounds to counteract trypanosomiasis, malaria, and Leishmaniasis are limited by the ability of the parasites to develop resistance to them and by their significant toxicity to the infected host. Therefore, there is an interest in developing new drugs that will interfere with the infectious life cycle of a parasite.

Pharmaceutical compounds having a semicarbazone scaffold have been evaluated for clinical use as an antihypertensive (Warren, J. D. et al., J. Med. Chem., 20:1520-1521 (1977)), anticonvulsant (Dimmock, J. R. et al., Epilepsia, 35:648-655 (1994); Pandeya, S. N. et al., Pharmacol Res., 37:17-22 (1998); Dimmock, J. R. et al., Drug Dev Res., 46:112-125 (1999)), and antiallodynic agent (Carter, R. B. et al., Proceeding, International Symposium “Ion Channels in Pain and Neuroprotection” March 14-17, San Francisco, Calif.; p 19 (1999)). For example, the semicarbazone compound 4-[4-fluorophenoxy]benzaldehyde semicarbazone has entered clinical trials for the treatment of neuropathic pain (Ramu, K. et al., Drug Metab. Dispos., 28:1153-1161 (2000)). Recently, 5-nitrofurfural N-butyl semicarbazone (Cerecetto. H. et al., Farmaco, 53:89-94 (1998); Cerecetto, H. et al., J. Med. Chem., 42:1941-1950 (1999); Cerecetto, H. et al., Eur. J. Med. Chem., 35:343-350 (2000)) has been shown to have antiTrypanosomal activities targeting trypanothione reductase through a nitro anion radical mechanism, however, no clear target validation was reported in these papers.

Therefore, there is a pressing interest in developing potent, efficacious, economically synthesized pharmaceutical compounds with minimal toxicity and maximal bioavailability for the effective treatment of these infectious parasitic diseases.

### SUMMARY OF THE INVENTION

The present invention relates to a novel class of compounds that function as anti-parasitic agents and the use of such compounds in methods of treating and preventing protozoan infections. The compounds also find use in inhibiting cellular replication associated with malignancy of cancer cells.

In a first aspect, the present invention provides anti-parasitic compounds having the formula:

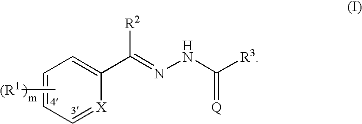

In Formula (I), Q is selected from ═S and ═O, m is an integer from 1 to 3, and X is selected from ═CH— and ═N—.

R¹ is independently selected from hydrogen, halogen, unsubstituted alkyl, substituted (C₃-C₁₀) alkyl, substituted or unsubstituted heteroalkyl with a carbon atom point of attachment, substituted or unsubstituted heterocycloalkyl, substituted or unsubstituted cycloalkyl, substituted or unsubstituted aryl, substituted or unsubstituted heteroaryl, —OR^{1A}, and —NR^{1B}R^{1C}.

R^{1A} is selected from (C₃-C₁₀) unsubstituted alkyl, substituted alkyl, unsubstituted C₃-C₁₀ alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. In addition, R^{1A} may be hydrogen if m is 1.

R^{1B} and R^{1C} are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

R² is selected from substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

R³ is selected from substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —SR^{3A}, and —NR^{3B}R^{3C}.

R^{3A} is selected from substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

R^{3B} and R^{3C} are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. R^{3B} and R^{3C} are optionally joined together to form a substituted or unsubstituted ring with the nitrogen to which they are attached.

In some embodiments where m is 1 and R¹ is Br, then at least one of R^{3B} and R^{3C} is selected from substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. In other embodiments where R¹ is hydrogen and Q is ═S, then R³ is not —NR^{3B}R^{3C}.

In another aspect, the present invention provides anti-parasitic compounds having the formula:

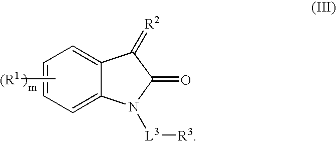

In Formula (III), m is an integer from 1 to 3.

R¹ is selected from hydrogen, halogen, —NH₂, —OH, —SO₂NHR^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. R^{1A} is selected from hydrogen, halogen, —OH, —NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl

R² is selected from ═O and ═N—NH—C(Q)-NR^{2A}R^{2B}. Q may be ═S or ═O. R^{2A} and R^{2B} are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. R^{2A} and R^{2B} are optionally joined together to form a ring with the nitrogen to which they are attached.

L³ is a member selected from substituted or unsubstituted alkylene, substituted or unsubstituted heteroalkylene, substituted or unsubstituted cycloalkylene, substituted or unsubstituted heterocycloalkylene, substituted or unsubstituted arylene, and substituted or unsubstituted heteroarylene.

R³ is selected from substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl, and NR^{3A}R^{3B}. R^{3A} and R^{3B} are members independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. R^{3A} and R^{3B} are optionally joined together to form a ring with the nitrogen to which they are attached.

In another aspect, the present invention provides anti-parasitic compounds having the formula:

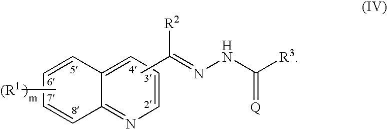

In Formula (IV), Q is selected from ═S and ═O and m is an integer from 1 to 6.

R¹ is independently selected from hydrogen, halogen, substituted or unsubstituted alkyl, substituted or unsubstituted heterocycloalkyl, substituted or unsubstituted cycloalkyl, substituted or unsubstituted aryl, substituted or unsubstituted heteroaryl, —OR^{1A}, and —NR^{1B}R^{1C}.

R^{1A} is selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

R^{1B} and R^{1C} are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

R² is substituted or unsubstituted alkyl.

R³ is selected from substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —SR^{3A}, and —NR^{3B}R^{3C}.

R^{3A} is selected from substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

R^{3B} and R^{3C} are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. R^{3B} and R^{3C} are optionally joined together to form a substituted or unsubstituted ring with the nitrogen to which they are attached.

In another aspect, the present invention provides anti-parasitic compounds having the formula:

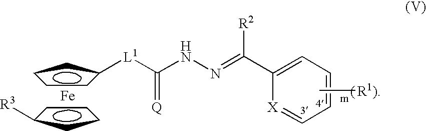

In Formula (V), Q is selected from ═S and ═O, m is an integer from 1 to 3, and X is a member selected from ═CH— and ═N—.

L¹ is selected from substituted or unsubstituted alkylene, substituted or unsubstituted heteroalkylene, substituted or unsubstituted cycloalkylene, substituted or unsubstituted heterocycloalkylene, substituted or unsubstituted arylene, and substituted or unsubstituted heteroarylene.

R¹ is independently selected from hydrogen, halogen, substituted or unsubstituted alkyl, substituted or unsubstituted heterocycloalkyl, substituted or unsubstituted cycloalkyl, substituted or unsubstituted aryl, substituted or unsubstituted heteroaryl, —OR^{1A}, and —NR^{1B}R^{1C}.

R^{1A} is selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

R^{1B} and R^{1C} are members independently selected from hydrogen, substituted or unsubstituted allyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

R² is substituted or unsubstituted alkyl.

R³ is selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

In another aspect, the present invention provides anti-parasitic compounds having the formula:

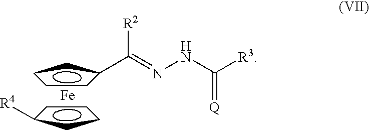

In Formula (VII), Q is selected from ═S and ═O.

R² is substituted or unsubstituted alkyl.

R³ is selected from substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —SR^{3A}, and —NR^{3B}R^{3C}.

R^{3A} is selected from substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

R^{3B} and R^{3C} are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. R^{3B} and R^{3C} are optionally joined together to form a substituted or unsubstituted ring with the nitrogen to which they are attached.

R⁴ is selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

In another aspect, the present invention provides a pharmaceutical composition including a therapeutically effective-amount of an anti-parasitic compound of the present inventions and a physiologically acceptable carrier.

In another aspect, the present invention provides methods of treating or preventing a parasitic disease. The method includes the step of administering to a patient in need thereof a sufficient amount of a pharmaceutical composition of the present invention. The pharmaceutical compositions of the present invention include an anti-parasitic compound of the present invention. Thus, the parasitic disease is treated or prevented by contacting a compound of the present invention with a parasite. In some embodiments, the patient is human.

In another aspect, the present invention provides methods of treating or preventing cancer. The methods include the step of administering to a patient in need thereof a sufficient amount of a pharmaceutical composition including a compound of the present invention.

### BRIEF DESCRIPTION OF THE DRAWINGS

FIG. 1 shows the results of a structure activity relationship study against cruzain and T. cruzi.

FIG. 2 shows the results of a structure activity relationship study against cruzain and T. cruzi.

FIG. 3 shows the results of a structure activity relationship study against rhodesain and T. brucei.

FIG. 4 shows the results of a structure activity relationship study against rhodesain and T. brucei.

FIG. 5 shows the results of a structure activity relationship study against falcipain and P. falciparum.

FIG. 6 shows the results of a structure activity relationship study against falcipain and P. falciparum.

FIG. 7 shows a chart summarizing the results of toxicity studies for selected compounds.

FIG. 8 shows exemplary anti-parasitic compounds of the present invention.

FIG. 9 shows in vitro inhibition in T. Brucei and cytotoxicity data for selected compounds of the present invention.

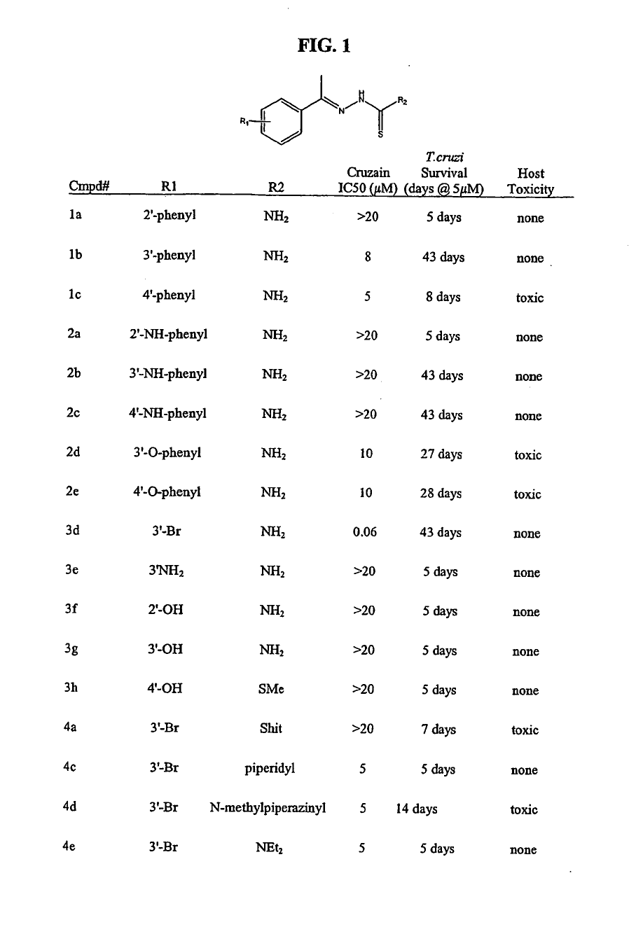

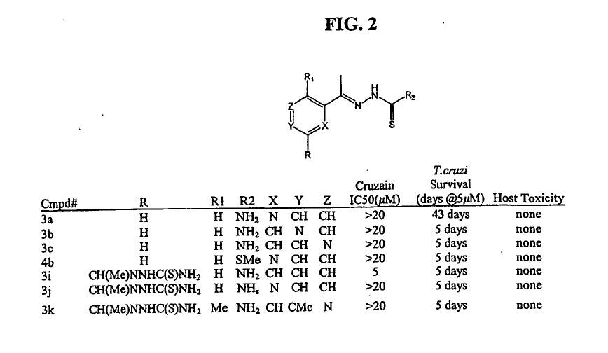

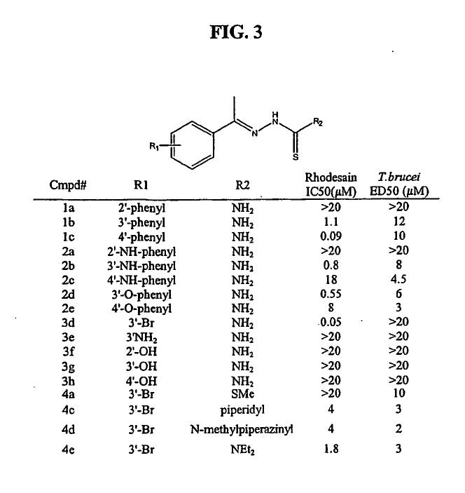

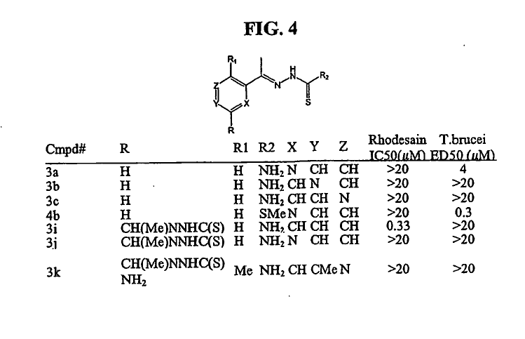

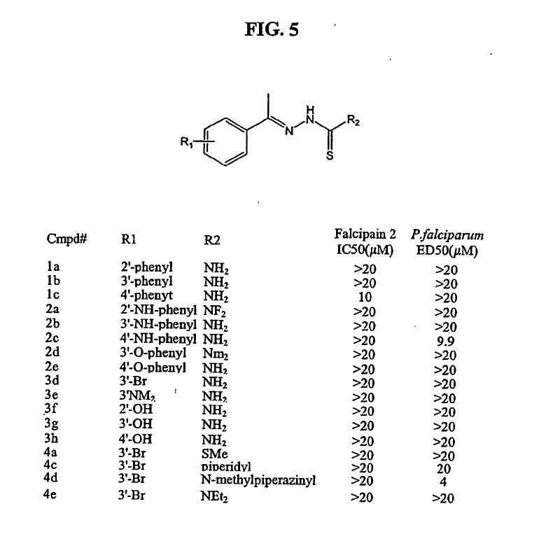

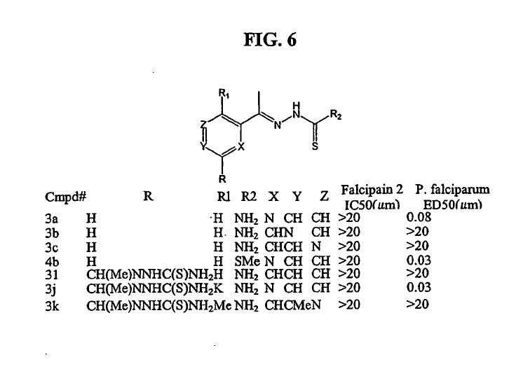

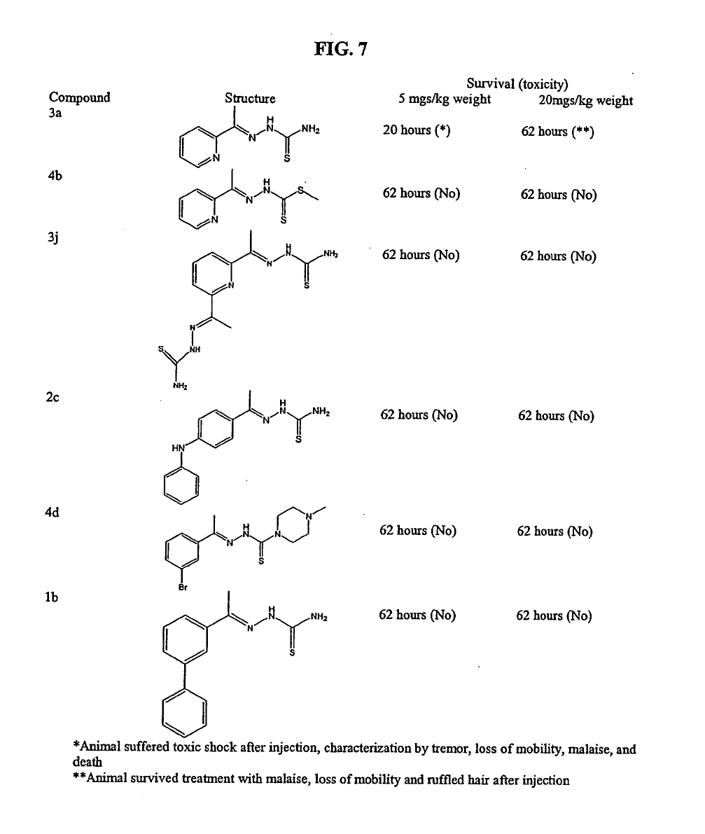

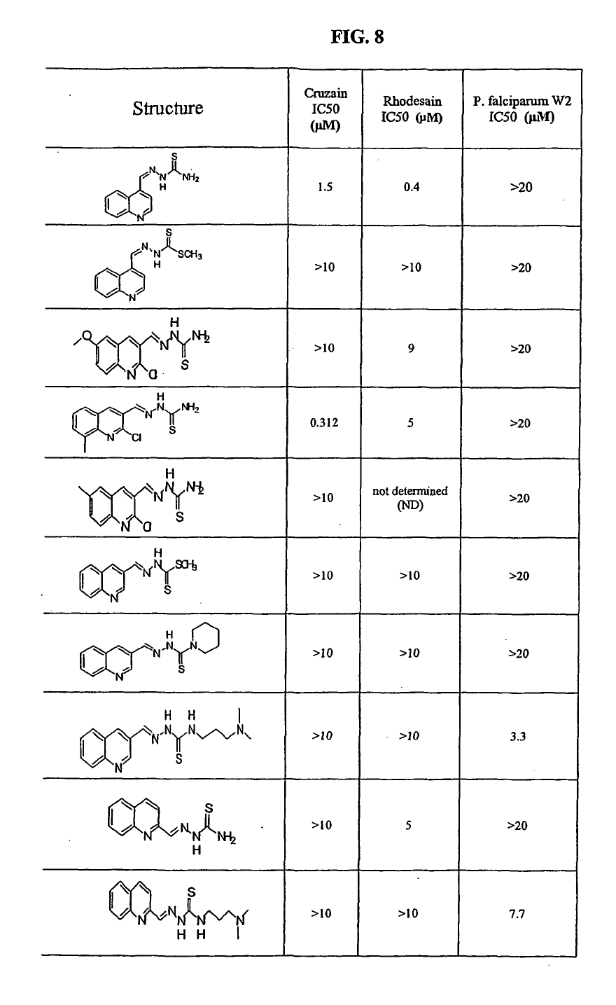

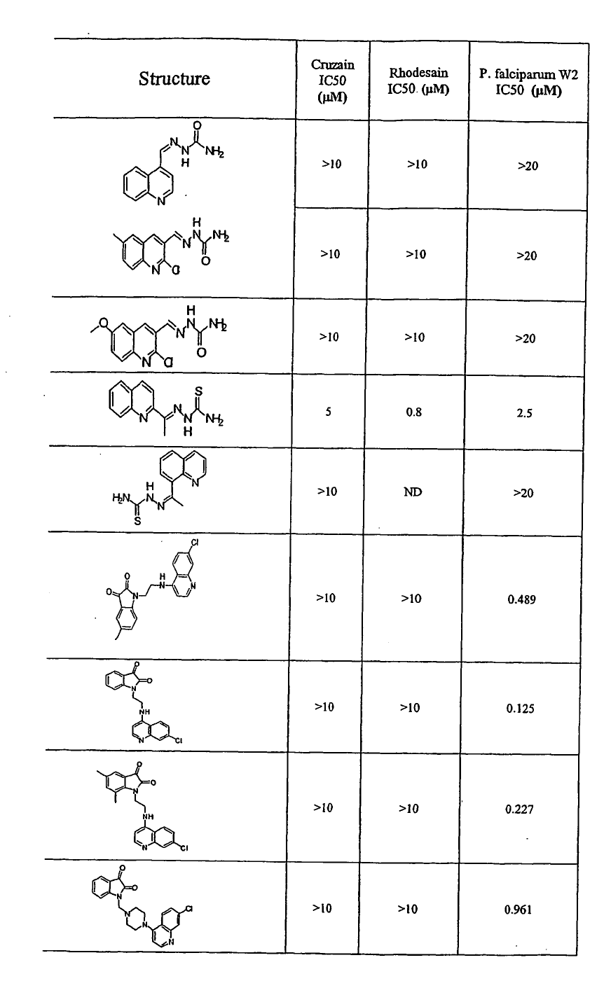

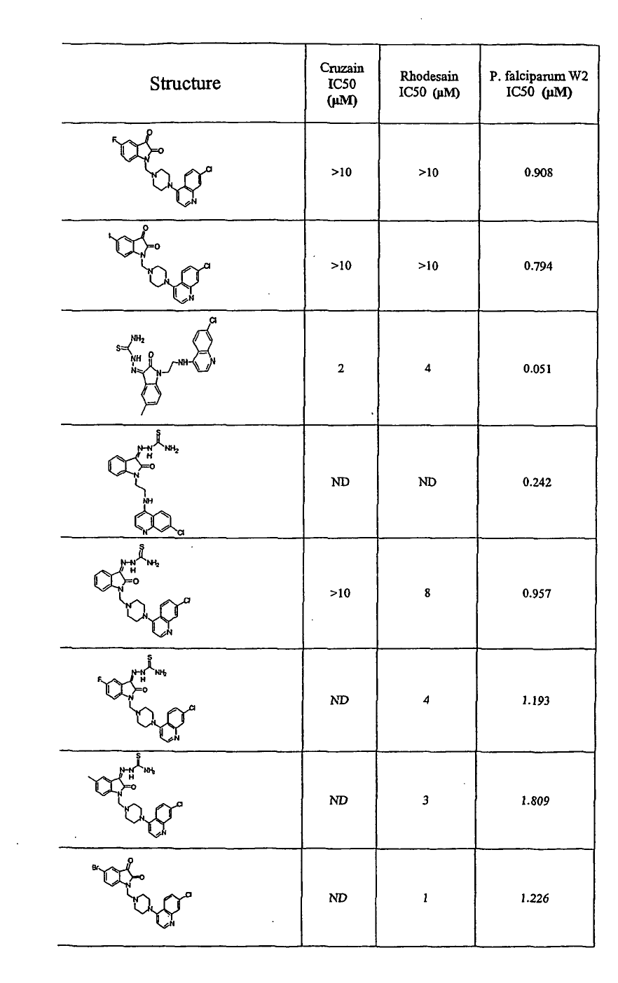

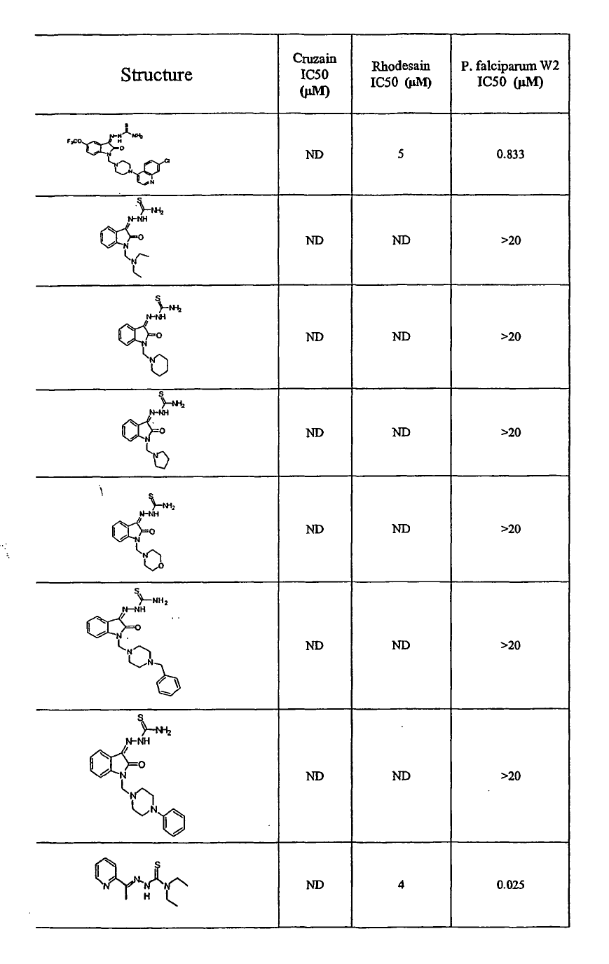

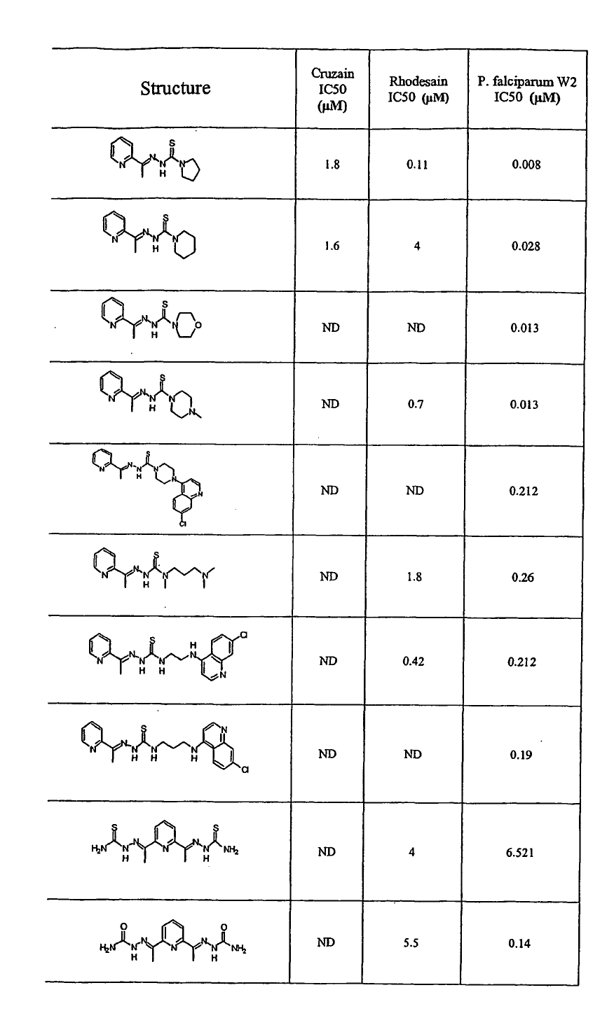

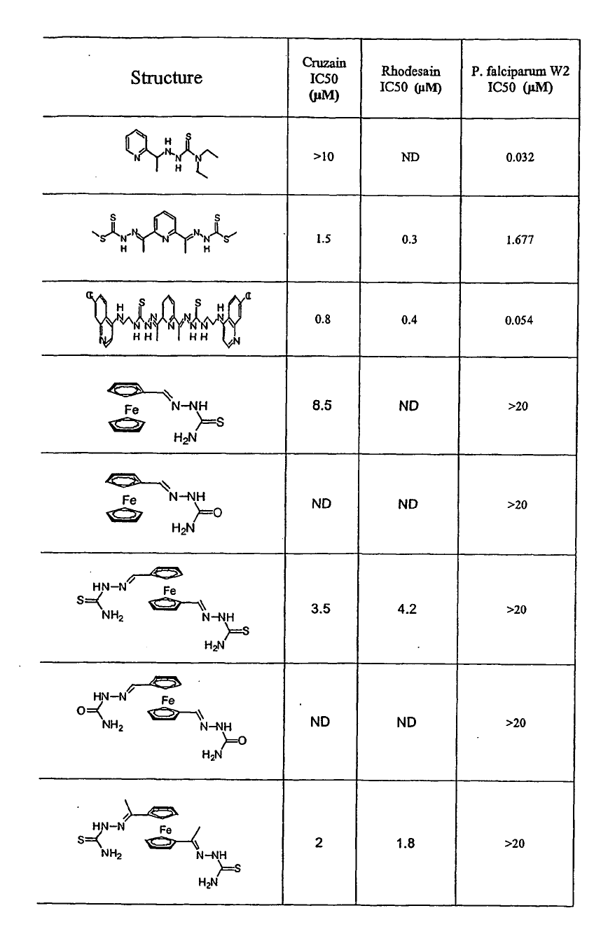

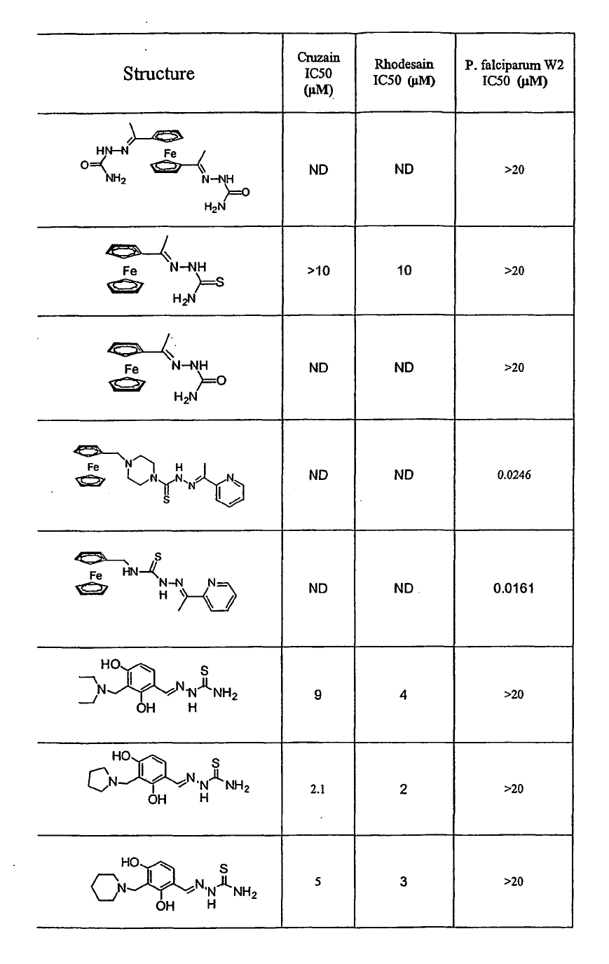

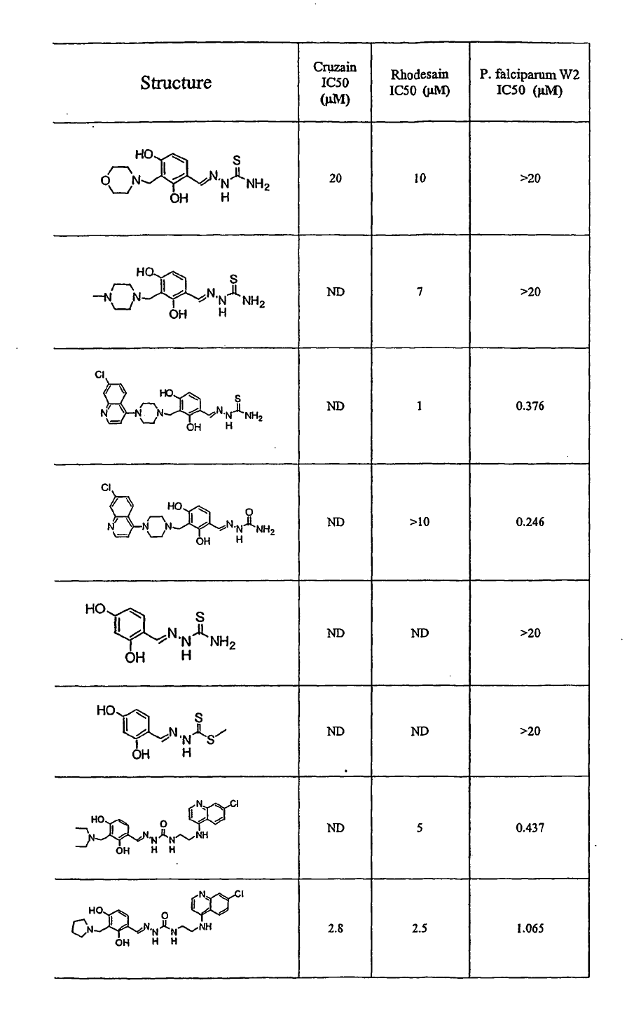

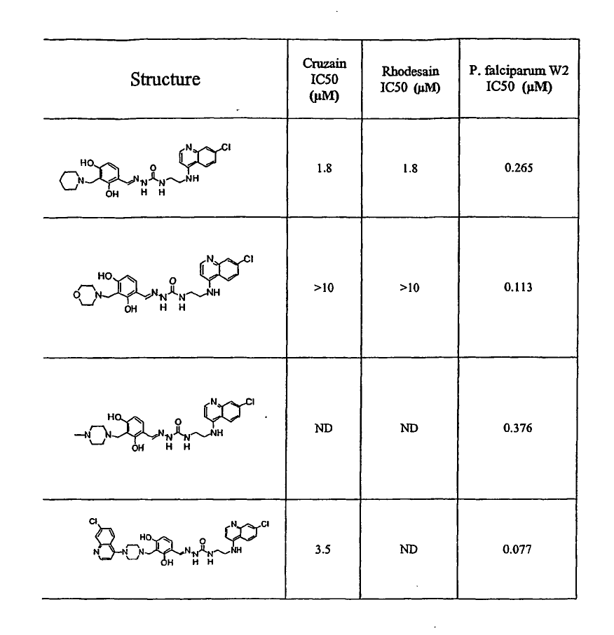

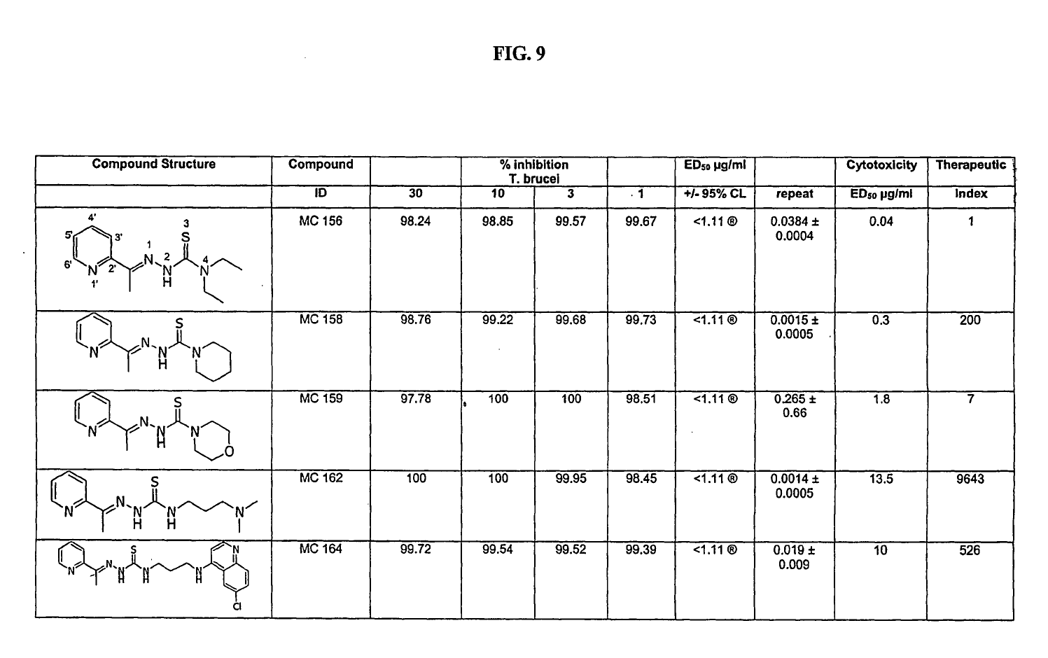

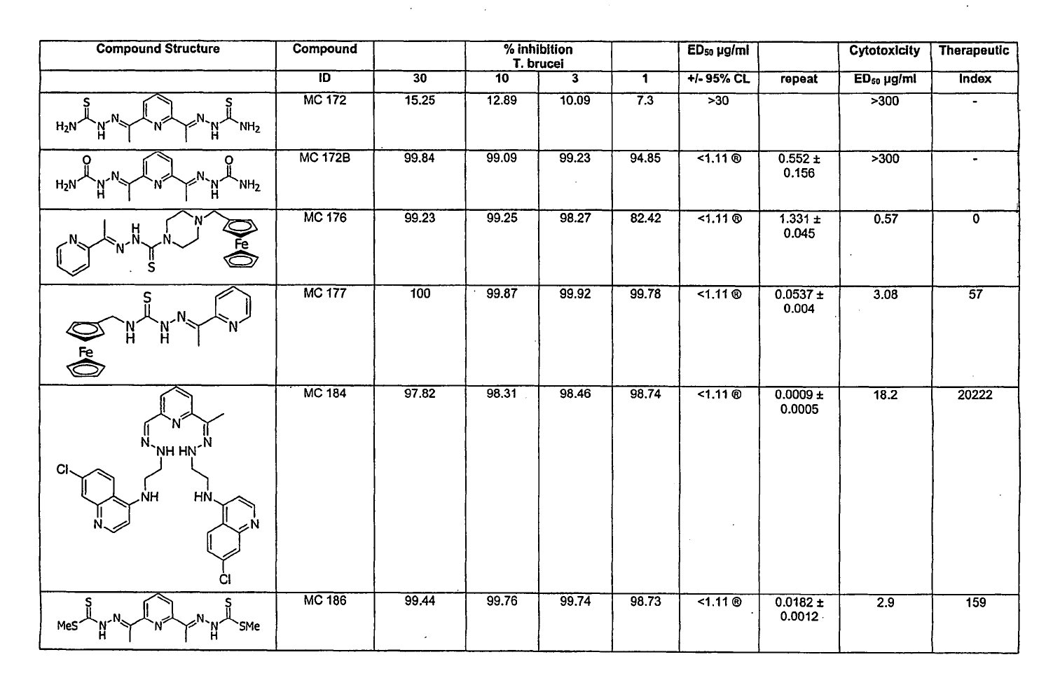

### DETAILED DESCRIPTION OF THE INVENTION

Introduction

Trypanosoma, Leishmania, Plasmodium and Trichomonas infections result in diseases affecting millions of people worldwide. In this application, we report a novel class of compounds that disrupt the parasitic infectious life cycle and serve as promising agents for anti-parasitic therapy. The advantages of the compounds are many, including (i) minimal cellular toxicity, (ii) physical properties compatible with desirable pharmacokinetics (low molecular weight, favorable C log P, favorable hydrogen bond donating and accepting capabilities), (iii) high potency of target inhibition, with IC₅₀ values at the low nanomolar level, (iv) parasiticidal and parasitistatic efficacy against parasite infections of cells, (v) efficient synthesis and inexpensive production, and (vi) improved bioavailability over peptidyl inhibitors. The parasiticidal activity of the compounds of the present invention represents a significant advance.

Definitions

By “parasitistatic” or “trypanostatic” or “Plasmodium-static” or “Leishmania-static” or “Trichomonas-static” is intended that the intracellular cycle of the parasite is completed at a slower growth rate and the infected host cells survive longer.

The term “parasiticidal” or “trypanocidal” or “Plasmodium-cidal” or “Leishmania-cidal” or “trichamonacidal” means that the intracellular cycle of the parasite is not completed leading to the death of the parasite. Anti-parasitic compounds of the present invention are parasiticidal. Similarly, anti-Trypanosoma, anti-Plasmodium, anti-Leishmania, and anti-Trichomonas compounds of the present invention are “trypanocidal,” “Plasmodium-cidal,”“Leishmania-cidal,” and “trichamonacidal,” respectively.

For the compounds of the invention, the term “alkyl,” by itself or as part of another substitutent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e., C1-C10 means one to ten carbons). Examples of saturated hydrocarbon radicals include groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below as “heteroalkyl.” Alkyl groups which are limited to hydrocarbon groups are termed “homoalkyl”.

The term “alkylene” by itself or as part of another substitutent means a divalent radical derived from an alkane, as exemplified by —CH2CH2CH2CH2-, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Examples include —CH2-CH2-O—CH3, —CH2-CH2-NH—CH3, —CH2-CH2-N(CH3)-CH3, —CH2-S—CH2-CH3, —CH2-CH2, —S(O)—CH3, —CH2-CH2-S(O)2-CH3, —CH═CH—O—CH3, —Si(CH3)3, —CH2-CH═N—OCH3, and —CH═CH—N(CH3)-CH3. Up to two heteroatoms may be consecutive, such as, for example, —CH2-NH—OCH3 and —CH2-O—Si(CH3)3. Similarly, the term “heteroalkylene” by itself or as part of another substitutent means a divalent radical derived from heteroalkyl, as exemplified by —CH2-CH2-S—CH2CH2- and —CH2-S—CH2-CH2-NH—CH2-. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include cyclopentyl, cyclohexyl, 1-cyclohexenyl, 3-cyclohexenyl, cycloheptyl, and the like. Examples of heterocycloalkyl include 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” by themselves or as part of another substitutent means a divalent radical derived from a cycloalkyl or heterocycloalkyl, respectively.

The terms “halo” or “halogen,” by themselves or as part of another substitutent, mean, unless otherwise stated, a fluorine, chlorine, bromine, or iodine atom. Additionally, terms such as “haloalkyl,” are meant to include monohaloalkyl and polyhaloalkyl. For example, the term “halo(C1-C4)alkyl” is mean to include trifluoromethyl, 2,2,2-trifluoroethyl, 4-chlorobutyl, 3-bromopropyl, and the like.

The term “aryl” means, unless otherwise stated, a polyunsaturated, typically aromatic, hydrocarbon substitutent which can be a single ring or multiple rings (up to three rings) which are fused together or linked covalently. The term “heteroaryl” refers to aryl groups (or rings) that contain from zero to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substitutents described below. The terms “arylene” and “heteroarylene” by themselves or as part of another substitutent means a divalent radical derived from an aryl or heteroaryl, respectively.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substitutents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be a variety of groups selected from: —OR′, ═O, ═NR′, ═N—OR′, —NR′R″, —SR′, -halogen, —SiR′R″R′″, —OC(O)R′, —C(O)R′, —CO2R′, —CONR′R″, —OC(O)NR′R″, —NR″C(O)R′, —NR′—C(O)NR″R′″, —NR″C(O)2R′, —NH—C(NH2)=NH, —NR′C(NH2)=NH, —NH—C(NH2)=NR′, —S(O)R′, —S(O)2R′, —S(O)2NR′R″, —CN and —NO2 in a number ranging from zero to (2m′+1), where m′ is the total number of carbon atoms in such radical. R′, R″ and R′″ each independently refer to hydrogen, unsubstituted (C1-C8)alkyl and heteroalkyl, unsubstituted aryl, aryl substituted with 1-3 halogens, unsubstituted alkyl, alkoxy or thioalkoxy groups, or aryl-(C1-C4)alkyl groups. When R′ and R″ are attached to the same nitrogen atom, they can be combined with the nitrogen atom to form a 5-, 6-, or 7-membered ring. For example, —NR′R″ is meant to include 1-pyrrolidinyl and 4-morpholinyl. From the above discussion of substitutents, one of skill in the art will understand that the term “alkyl” is meant to include groups such as haloalkyl (e.g., —CF3 and —CH2CF3) and acyl (e.g., —C(O)CH3, —C(O)CF3, —C(O)CH2OCH3, and the like).

Similarly, substitutents for the aryl and heteroaryl groups are varied and are selected from: -halogen, —OR′, —OC(O)R′, —NR′R″, —SR′, —R′, —CN, —NO2, —CO2R′, —CONR′R″, —C(O)R′, —OC(O)NR′R″, —NR″C(O)R′, —NR″C(O)2R′, —NR′—C(O)NR″R′″, —NH—C(NH2)=NH, —NR′C(NH2)=NH, —NH—C(NH2)=NR′, —S(O)R′, —S(O)2R′, —S(O)2NR′R″, —N3, —CH(Ph)2, perfluoro(C1-C4)alkoxy, and perfluoro(C1-C4)alkyl, in a number ranging from zero to the total number of open valences on the aromatic ring system; and where R′, R″ and R′″ are independently selected from hydrogen, (C1-C8)alkyl and heteroalkyl, unsubstituted aryl and heteroaryl, (unsubstituted aryl)-(C1-C4)alkyl, and (unsubstituted aryl)oxy-(C1-C4)alkyl.

Two of the substitutents on adjacent atoms of the aryl or heteroaryl ring may optionally be replaced with a substitutent of the formula -T-C(O)—(CH2)q-U—, wherein T and U are independently —NH—, —O—, —CH2- or a single bond, and q is an integer of from 0 to 2. Alternatively, two of the substitutents on adjacent atoms of the aryl or heteroaryl ring may optionally be replaced with a substitutent of the formula -A-(CH2)r-B—, wherein A and B are independently —CH2-, —O—, —NH—, —S—, —S(O)—, —S(O)2-, —S(O)2NR′— or a single bond, and r is an integer of from 1 to 3. One of the single bonds of the new ring so formed may optionally be replaced with a double bond. Alternatively, two of the substitutents on adjacent atoms of the aryl or heteroaryl ring may optionally be replaced with a substitutent of the formula —(CH2)s-X—(CH2)t-, where s and t are independently integers of from 0 to 3, and X is —O—, —NR′—, —S—, —S(O)—, —S(O)2-, or —S(O)2NR′—. The substitutent R′ in —NR′— and —S(O)2NR′— is selected from hydrogen or unsubstituted (C1-C6)alkyl.

As used herein, the term “heteroatom” is meant to include oxygen (O), nitrogen (N), sulfur (S) and silicon (Si).

The term “pharmaceutically acceptable salts” or “pharmaceutically acceptable carrier” is meant to include salts of the active compounds which are prepared with relatively nontoxic acids or bases, depending on the particular substitutents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are all intended to be encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

One of skill in the art will immediately recognize that where a substitutent is present in a general formula of a compound of the present invention, the substitutent may be rotated freely around the bond with which the substitutent is attached to the remainder of the molecule. Thus, where a 2-pyridinyl substitutent is present, as in the compounds of Formula (I), for example, one of skill will immediately recognize that the pyridinyl substitutent may be rotated to bring the ring nitrogen closer to the R² group.

A semicarbazone substitutent of the present invention has the general formula —CHR′—N═NH—C(Q)-R″, where R′ and R″ may be independently selected from substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

Anti-Parasitic Compounds

In a first aspect, the present invention provides anti-parasitic compounds having the formula:

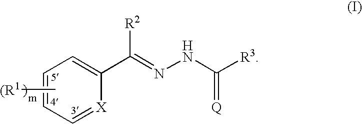

In Formula (I), m is an integer from 1 to 3 and X is selected from ═CH— and ═N—. Q is selected from ═S and ═O.

R¹ is independently selected from hydrogen, halogen, unsubstituted alkyl, substituted (C₃-C₁₀) alkyl, substituted or unsubstituted heteroalkyl with a carbon atom point of attachment, substituted or unsubstituted heterocycloalkyl, substituted or unsubstituted cycloalkyl, substituted or unsubstituted aryl, substituted or unsubstituted heteroaryl, —OR^{1A}, and —NR^{1B}R^{1C}. R¹ may also be selected from unsubstituted alkyl, substituted or unsubstituted heterocycloalkyl, substituted or unsubstituted cycloalkyl, substituted or unsubstituted aryl, substituted or unsubstituted heteroaryl, —OR^{1A}, and —NR^{1B}R^{1C}.

R^{1A} is selected from (C₃-C₁₀) unsubstituted alkyl, substituted alkyl, unsubstituted C₃-C₁₀ alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. In addition, R^{1A} may be hydrogen if m is 1 or 3. R^{1A} may be hydrogen if m is 2, and the second R¹ is not an alkyloxy.

R^{1B} and R^{1C} are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. R^{1B} and R^{1C} are optionally joined together to form a substituted or unsubstituted ring with the nitrogen to which they are attached.

R² is selected from substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. In some embodiments, R² is a substituted or unsubstituted alkyl.

R³ is selected from substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —SR^{3A}, and —NR^{3B}R^{3C}.

R^{3A} is selected from substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

R^{3B} and R^{3C} are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. R^{3B} and R^{3C} are optionally joined together to form a substituted or unsubstituted ring with the nitrogen to which they are attached.

In some embodiments where m is 1 and R¹ is Br, then at least one of R^{3B} and R^{3C} is selected from substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. In other embodiments where R¹ is hydrogen and Q is ═S, then R³ is selected from substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —SR^{3A}. In still other embodiments, where R¹ is hydrogen, X is ═N—.

The integer m may also be selected from 1 and 2. In some anti-parasitic compounds of the present invention, m is 1.

A variety of R¹ groups are useful in the anti-parasitic compounds of Formula (I). Exemplary R¹ groups include hydrogen, Br, substituted or unsubstituted C₁-C₁₀ alkyl, substituted or unsubstituted 2 to 10 membered heteroalkyl, substituted or unsubstituted 5 to 7 membered heterocycloalkyl, substituted or unsubstituted aryl, —OR^{1A}, and —NR^{1B}R^{1C}.

In a related embodiment, R¹ is -L¹NNHC(S)NH₂. L¹ is a member selected from substituted or unsubstituted alkylene and substituted or unsubstituted heteroalkylene. In a further related embodiment, L¹ is a substituted or unsubstituted C₁-C₅ alkylene, such as —CH(CH₃)—.

R¹ may also be selected from hydrogen, substituted or unsubstituted phenyl, substituted or unsubstituted —NH-phenyl, substituted or unsubstituted —O-phenyl, and substituted or unsubstituted quinolinyl.

Exemplary R¹ substituted or unsubstituted heterocycloalkyl groups include substituted or unsubstituted pyrrolidinyl, substituted or unsubstituted piperidyl, substituted or unsubstituted piperizyl, substituted or unsubstituted morpholinyl, and substituted or unsubstituted pyridyl. Where R¹ is a substituted heterocycloalkyl, the heterocycloalkyl substitutent may be selected from substituted or unsubstituted C₁-C₅ alkyl, substituted or unsubstituted aryl, and substituted or unsubstituted heteroaryl, including substituted or unsubstituted quinolinyl.

In some embodiments, R¹ does not include a halogen. In other embodiments, R¹ is not —OCH₃.

R¹ may be attached to the 3′-position or the 4′-position. Exemplary substitutents attached to the 3′-position or the 4′-position include 3′-NH-phenyl, 4′-NH-phenyl, 3′-O-phenyl, 4′-O-phenyl, 3′-phenyl, and 4′-phenyl.

As discussed above, R^{1A} may be hydrogen if m is 1 or 3. In addition, R^{1A} may be hydrogen if m is 2, and the second R¹ is not an alkyloxy. In an exemplary embodiment, the compound of Formula (I) includes two R¹ hydroxyl groups. In a further embodiment, the two hydroxyl groups are attached at the 3′ position and the 5′ position. The compound may further include a third R¹ group at the 4′ position, such as —NR^{1B}R^{1C}.

R^{1B} may simply be hydrogen. R^{1C} may be substituted or unsubstituted aryl.

Alternatively, R¹ may also be a substituted or unsubstituted thiosemicarbazone or substituted or unsubstituted semicarbazone. In an exemplary embodiment, R¹ has the formula 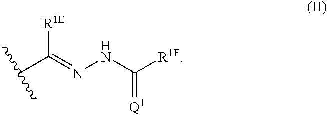
In Formula (II), Q¹, R^{1E}, and R^{1F} are equivalent to Q, R² and R³ as defined above in Formula (I).

In some embodiments, R² is an unsubstituted alkyl, such as an unsubstituted C₁-C₁₀ alkyl. Thus, R² may simply be a methyl group.

Useful R³ groups include substituted or unsubstituted C₁-C₁₀ alkyl, substituted or unsubstituted 2 to 10 membered heteroalkyl, substituted or unsubstituted 5 to 7 membered heterocycloalkyl (e.g. piperazine and piperidine), substituted or unsubstituted heteroaryl, —SR^{3A}, and —NR^{3B}R^{3C}. In some embodiments, R^{3A} is substituted or unsubstituted (C₁-C₅) alkyl.

R^{3B} and R^{3C} may independently be selected from hydrogen, substituted or unsubstituted alkyl, and substituted or unsubstituted heteroalkyl. In a related embodiment, R^{3B} and R^{3C} are independently be selected from hydrogen, substituted or unsubstituted C₁-C₁₀ alkyl, and substituted or unsubstituted 2 to 10 membered heterocycloalkyl. In a further related embodiment, the 2 to 10 membered heteroalkyl is a substituted or unsubstituted alkylamine. The substituted alkylamine may include a variety of substitutents, including substituted or unsubstituted aryls and substituted or unsubstituted heteroaryls, such as substituted or unsubstituted quinolinyl. For example, the substituted alkylamine may have the formula —(CH₂)ₙNR^{3B1}R^{3B2}, where n is an integer from 1 to 8.

R^{3B1} and R^{3B2} are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. In a related embodiment, R^{3B1} and R^{3B2} are independently selected from substituted or unsubstituted C₁-C₅ alkyl, substituted or unsubstituted aryl, and substituted or unsubstituted heteroaryl. In a further related embodiment, R^{3B1} is substituted or unsubstituted quinolinyl, such as a 7-halo-3-quinolinyl.

R^{3B} and R^{3C} may optionally be joined together to form a substituted or unsubstituted ring with the nitrogen to which they are attached. The ring formed by R^{3B} and R^{3C} may be selected from substituted or unsubstituted piperidyl, substituted or unsubstituted piperizyl, substituted or unsubstituted morpholinyl, substituted or unsubstituted pyrrolidinyl, substituted or unsubstituted quinolinyl, and substituted or unsubstituted pyridyl. The ring formed by R^{3B} and R^{3C} may be substituted with a wide variety of substitutents, including substituted or unsubstituted (C₁-C₅) alkyl, substituted or unsubstituted aryl, and substituted or unsubstituted heteroaryl. In an exemplary embodiment, the ring substitutent is substituted or unsubstituted quinolinyl.

In some embodiments, R^{3C} is unsubstituted (C₁-C₅) allyl.

In another exemplary embodiment of the compound of Formula (I), m is 1. R¹ is selected from hydrogen, Br, substituted or unsubstituted aryl, —OR^{1A}, —NR^{1B}R^{1C}, and -L¹NNHC(S)NH₂. R^{1A} is substituted or unsubstituted aryl, R^{1B} is hydrogen, R^{1C} is substituted or unsubstituted aryl, and L¹ is selected from substituted or unsubstituted alkylene and substituted or unsubstituted heteroalkylene. R² is methyl and R³ is selected from —SR^{3A} and —NR^{3B}R^{3C}. R^{3A} is selected from substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. R^{3B} and R^{3C} are independently selected from hydrogen, substituted or unsubstituted alkyl. R^{3B} and R^{3C} are optionally joined together to form a substituted or unsubstituted ring with the nitrogen to which they are attached. The ring formed by R^{3B} and R^{3C} is selected from substituted or unsubstituted piperidyl, substituted or unsubstituted piperizyl, and substituted or unsubstituted pyridyl.

Alternatively, the anti-parasitic compound may be an embodiment of Formula (I) wherein m is 1 and R¹ is attached to the 3′-position or the 4′-position. R¹ is selected from hydrogen, substituted or unsubstituted phenyl, substituted or unsubstituted —NH-phenyl, and substituted or unsubstituted —O-phenyl. R² is methyl and R³ is selected from —NH₂, substituted or unsubstituted piperidyl, substituted or unsubstituted piperazinyl, —SR^{3A}, and NR^{3B}R^{3C}. R^{3A} and R^{3C} are substituted or unsubstituted (C₁-C₅) alkyl.

In a related embodiment, R³ is selected from substituted or unsubstituted piperidyl, and substituted or unsubstituted piperazinyl. R^{3A} and R^{3C} are substituted or unsubstituted (C₁-C₅) alkyl. In a further related embodiment, X is ═N—, R¹ is hydrogen, and R³ is —SR^{3A}.

In other embodiments, m is 1 and R¹ is selected from hydrogen, substituted or unsubstituted phenyl, substituted or unsubstituted —NH-phenyl, and substituted or unsubstituted —O-phenyl. R¹ is attached to the 3′-position or the 4′-position. R² is methyl and R³ is selected from —NH₂ and substituted or unsubstituted piperazinyl.

In a related embodiment, X is ═CH—, and R¹ is selected from 3′-NH-phenyl, 4′-NH-phenyl, 3′-O-phenyl, 4′-O-phenyl, and 3′-phenyl. In a further related embodiment, R¹ is selected from 3′-NH-phenyl, 4′-NH-phenyl, and 3′-phenyl.

In another exemplary embodiment, m is 1, X is ═CH—, and R¹ is selected from Br and substituted or unsubstituted 4′-NH-phenyl. R² is methyl and R³ is selected from —NH₂ and substituted or unsubstituted piperazinyl. Where R¹ is Br, then R³ is substituted or unsubstituted piperazinyl.

Alternatively, m is 1, X is ═N—, and R¹ is selected from hydrogen and -L¹NNHC(S)NH₂. L¹ is selected from substituted or unsubstituted alkylene and substituted or unsubstituted heteroalkylene. R² is methyl and R³ selected from —NH₂ and —SR^{3A}. R^{3A} is substituted or unsubstituted (C₁-C₅) alkyl.

In another aspect, the present invention provides anti-parasitic compounds having the formula:

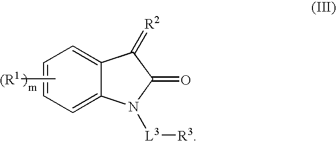

In Formula (III), m is an integer from 1 to 3.

R¹ is selected from hydrogen, halogen, —NH₂, —OH, —SO₂NHR^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. R^{1A} is selected from hydrogen, halogen, —OH, —NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl

R² is selected from ═O and ═N—NH—C(Q)-NR^{2A}R^{2B}. Q may be ═S or ═O. R^{2A} and R^{2B} are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. R^{2A} and R^{2B} are optionally joined together to form a ring with the nitrogen to which they are attached.

L³ is a member selected from substituted or unsubstituted alkylene, substituted or unsubstituted heteroalkylene, substituted or unsubstituted cycloalkylene, substituted or unsubstituted heterocycloalkylene, substituted or unsubstituted arylene, and substituted or unsubstituted heteroarylene.

R³ is selected from substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl, and NR^{3A}R^{3B}. R^{3A} and R^{3B} are members independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. R^{3A} and R^{3B} are optionally joined together to form a ring with the nitrogen to which they are attached.

In some embodiments, where R¹ is ═O, R³ is selected from substituted or unsubstituted quinolinyl.

A variety of R¹ groups are useful in the anti-parasitic compounds of Formula (III). Exemplary R¹ groups include hydrogen, halogen, and substituted or unsubstituted C₁-C₁₀ alkyl. In some related embodiments, the unsubstituted alkyl is a C₁-C₅ unsubstituted alkyl.

In some embodiments, L³ is a member selected from unsubstituted alkylene, unsubstituted heteroalkylene, and unsubstituted heterocycloalkylene.

R² may be an unsubstituted C₁-C₅ alkyl.

R³ may be selected from substituted or unsubstituted 5 to 7 membered heterocycloalkyl, substituted or unsubstituted aryl and substituted or unsubstituted heteroaryl. In a related embodiment, R³ is substituted or unsubstituted substituted quinolinyl. In a further related embodiment, R³ is unsubstituted quinolinyl, quinolinyl substituted with a halogen, substituted or unsubstituted piperidinyl, substituted or unsubstituted morpholinyl, substituted or unsubstituted piperazinyl, and —NR^{3A}R^{3B}. In a related embodiment, R^{3A} and R^{3B} are unsubstituted alkyl.

In another aspect, the present invention provides anti-parasitic compounds having the formula:

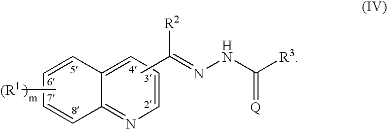

In Formula (IV), Q is selected from ═S and ═O and m is an integer from 1 to 6.

R¹ is independently selected from hydrogen, halogen, substituted or unsubstituted alkyl, substituted or unsubstituted heterocycloalkyl, substituted or unsubstituted cycloalkyl, substituted or unsubstituted aryl, substituted or unsubstituted heteroaryl, —OR^{1A}, and —NR^{1B}R^{1C}.

R^{1A} is selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

R^{1B} and R^{1C} are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

R² is substituted or unsubstituted alkyl.

R³ is selected from substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —SR^{3A}, and —NR^{3B}R^{3C}.

R^{3A} is selected from substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

R^{3B} and R^{3C} are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. R^{3B} and R^{3C} are optionally joined together to form a substituted or unsubstituted ring with the nitrogen to which they are attached.

In some embodiments where R¹ is hydrogen and Q is ═S, then only hydrogen or R¹ is attached to the 2′ position:

The compounds of Formula (IV) include a quinoline core, from 1 to 6 R¹ substitutents, and a semicarbazone substitutent (—CHR²—N═NH—C(Q)-R³). The R¹ substitutents and the semicarbazone substitutent may be attached to the quinoline core at any of the numbered positions denoted in Formula (IV). Thus, the R¹ substitutents and the semicarbazone substitutent may be attached to the quinoline core at a position selected from the 2′-position, 3′-position, 4′-position, 5′-position, 6′-position, 7′-position, and 8′-position. In some embodiments, the semicarbazone substitutent is attached to the quinoline core at a position selected from the 3′-position, 4′-position, and 8′-position. R¹ substitutents may be attached to the quinoline core at a position selected from the 6′-position and 8′-position.

A variety of R¹ substitutents are useful in the compounds of Formula (III). Exemplary R¹ groups include hydrogen, halogen, substituted or unsubstituted C₁-C₁₀ alkyl, and substituted or unsubstituted 5 to 7 membered heterocycloalkyl. In a related embodiment, R¹ is selected from hydrogen, halogen, unsubstituted C₁-C₅ alkyl, and substituted or unsubstituted 5 to 7 membered heterocycloalkyl.

R² may be an unsubstituted C₁-C₅ alkyl.

R³ may be selected from substituted or unsubstituted 2 to 10 membered heteroalkyl, substituted or unsubstituted 5 to 7 membered heterocycloalkyl, —SR^{3A}, and —NR^{3B}R^{3C}. In a related embodiment, R^{3A} is a C₁-C₅ unsubstituted alkyl. In another related embodiment, R^{3B} and R^{3C} are selected from hydrogen, substituted or unsubstituted C₁-C₅ alkyl, substituted or unsubstituted 2-5 membered heteroalkyl, and substituted or unsubstituted 5 to 6 membered heterocycloalkyl. In a further related embodiment, the 5 to 6 membered heterocycloalkyl includes at least one ring nitrogen (e.g. a piperidine ring). Alternatively, R^{3B} and R^{3C} are hydrogen.

In another aspect, the present invention provides anti-parasitic compounds having the formula:

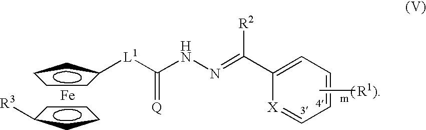

In Formula (V), X is a member selected from ═CH— and ═N—. Q is selected from ═S and ═O. The symbol m is an integer from 1 to 3.

L¹ is selected from substituted or unsubstituted alkylene, substituted or unsubstituted heteroalkylene, substituted or unsubstituted cycloalkylene, substituted or unsubstituted heterocycloalkylene, substituted or unsubstituted arylene, and substituted or unsubstituted heteroarylene.

R¹ is independently selected from hydrogen, halogen, substituted or unsubstituted alkyl, substituted or unsubstituted heterocycloalkyl, substituted or unsubstituted cycloalkyl, substituted or unsubstituted aryl, substituted or unsubstituted heteroaryl, —OR^{1A}, and —NR^{1B}R^{1C}.

R^{1A} is selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

R^{1B} and R^{1C} are members independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

R² is substituted or unsubstituted alkyl.

R³ is selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

A variety of L¹ groups are useful in the anti-parasitic compounds of Formula (V). Exemplary L¹ groups include substituted or unsubstituted C₁-C₁₀ alkylene, substituted or unsubstituted 1 to 10 membered heteroalkylene, and substituted or unsubstituted heterocycloalkylene. In a related embodiment, L¹ is selected from unsubstituted C₁-C₅ alkylene, unsubstituted 1 to 10 membered heteroalkylene, and unsubstituted 5 to 7 membered heterocycloalkylene.

Exemplary R¹ groups include hydrogen, halogen, substituted or unsubstituted C₁-C₁₀ alkyl, and substituted or unsubstituted 2 to 10 membered heterocycloalkyl.

R² may be an unsubstituted C₁-C₅ alkyl.

R³ may be selected from hydrogen, substituted or unsubstituted alkyl, substituted or unsubstituted heteroalkyl, and substituted or unsubstituted heterocycloalkyl. In a related embodiment, R³ is selected from hydrogen, substituted or unsubstituted C₁-C₁₀ alkyl, substituted or unsubstituted 2 to 10 membered heteroalkyl, and substituted or unsubstituted 5 to 7 membered heterocycloalkyl.

In some embodiments, R³ is equivalent to the semicarbazone moiety of Formula (V). Thus, R³ may have the formula

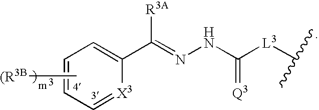

In Formula (VI), Q³, m³, X³, L³, R^{3A}, and R^{3B} are the equivalent to Q, m, X L¹, R¹, and R² in Formula (V) above.

In another aspect, the present invention provides anti-parasitic compounds having the formula:

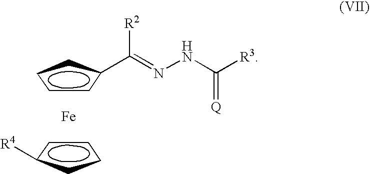

In Formula (VII), Q is selected from ═S and ═O.

R² is substituted or unsubstituted alkyl.

R³ is selected from substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —SR^{3A}, and —NR^{3B}R^{3C}.

R^{3A} is selected from substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

R^{3B} and R^{3C} are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. R^{3B} and R^{3C} are optionally joined together to form a substituted or unsubstituted ring with the nitrogen to which they are attached.

R⁴ is selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

In some embodiments where R⁴ is hydrogen, then R³ is not —NR^{3B}R^{3C}.

Exemplary R³ groups include from substituted or unsubstituted heteroalkyl, substituted or unsubstituted heterocycloalkyl, —SR^{3A}, and —NR^{3B}R^{3C}. In a related embodiment, R^{3A} is unsubstituted C₁-C₅ alkyl. In another related embodiment, R^{3B} and R^{3C} are selected from hydrogen, substituted or unsubstituted C₁-C₅ alkyl, substituted or unsubstituted 2-5 membered heteroalkyl, and a 5 to 6 membered heterocycloalkyl. In a further related embodiment, the 5 to 6 membered heterocycloalkyl includes at least one ring nitrogen (e.g. a piperidine ring).

R² may be an unsubstituted C₁-C₅ alkyl.

R⁴ may be selected from hydrogen, substituted or unsubstituted C₁-C₁₀ alkyl, and substituted or unsubstituted 2 to 10 membered heteroalkyl. In some embodiments, R³ is equivalent to the semicarbazone moiety of Formula (VII). Thus, R³ may have the formula

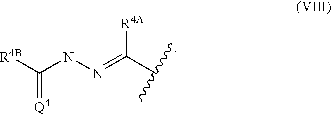

In Formula (VIII), Q⁴, R^{4A}, and R^{4B} are the equivalent to Q, R², and R³ in Formula (VII) above.

Exemplary Syntheses

Biphenyl thiosemicarbazones 1a-1c were prepared in high yield (80-90%) from the biphenylacetophenones, which were in turn prepared in good yield (60-70%) (Scheme I) via a Suzuki cross coupling procedure from the corresponding bromoacetophenones and phenylboronic acid.

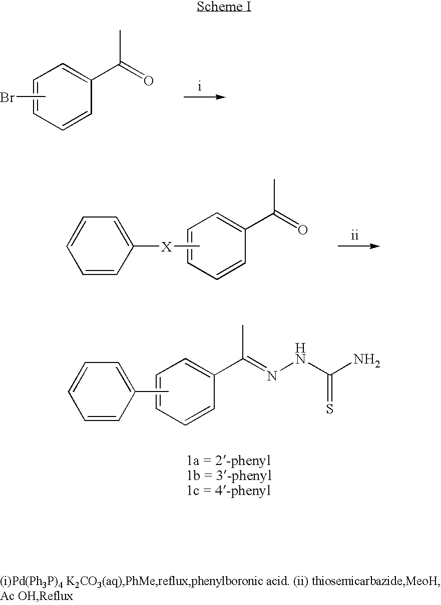

The biaryl amine and ether derivatives 2a-2e were also prepared in high (80-90%) via moderate yielding (50-60%) N- and O-arylation reactions, respectively, with phenylboronic acid and cupric acetate (Scheme II).

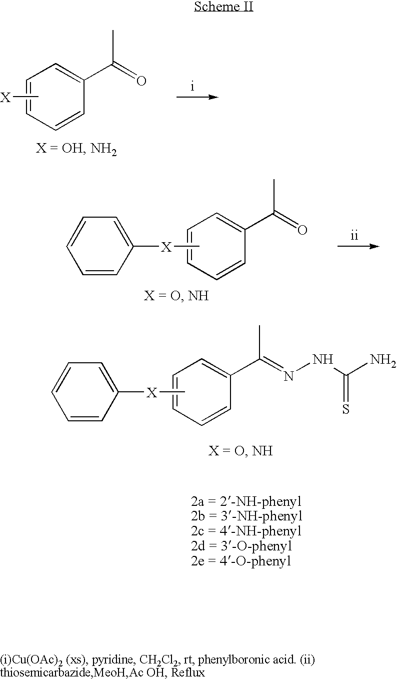

Treatment of the intermediate biphenyl, biaryl amine and biaryl ether acetophenones with thiosemicarbazide afforded compounds in high yield (80-90%). Thiosemicarbazones 3a-3h were prepared in high yield (80-90%) from commercially available starting materials by reaction with thiosemicarbazide (Scheme III).

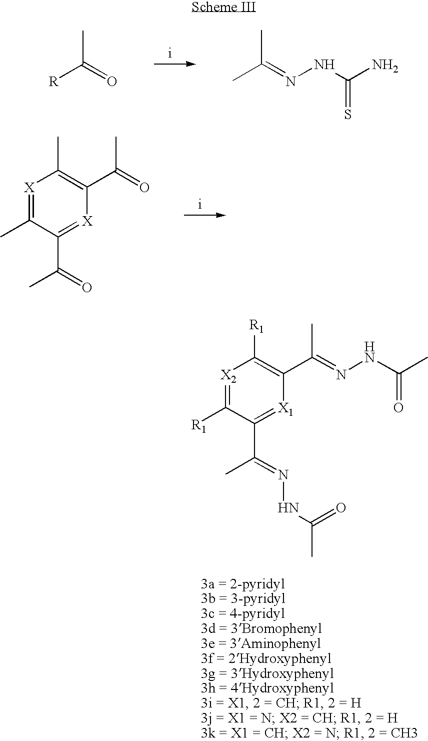

N,N-disubstituted derivatives 4a-4e were synthesized by reacting thiosemicarbazone thioesters with selected secondary amines (Scheme IV). 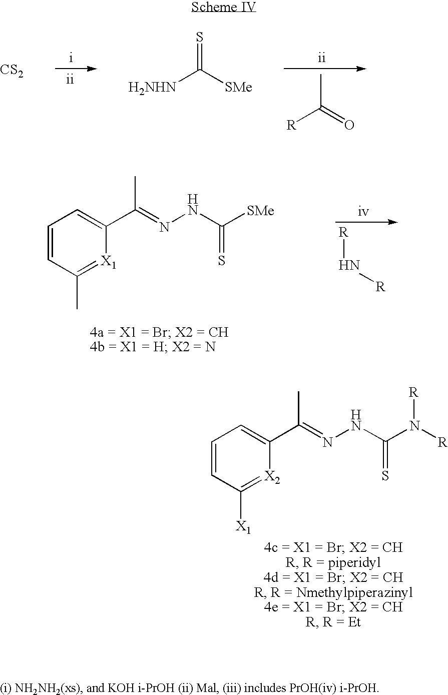
Administration and Pharmaceutical Compositions

In another aspect, the present invention provides a pharmaceutical composition including a therapeutically effective amount of a compound of the present inventions and a physiologically acceptable carrier.

Pharmaceutically and physiologically acceptable carriers are determined in part by the particular composition being administered (e.g., nucleic acid, protein, modulatory compounds or transduced cell), as well as by the particular method used to administer the composition. Accordingly, there are a wide variety of suitable formulations of pharmaceutical compositions of the present invention (see, e.g., Remington's Pharmaceutical Sciences, 17th ed., 1989). Suitable methods of administration include oral, nasal, rectal, and parenteral administration. Other delivery methods known to those of skill in the art can be used, e.g., liposomes, microspheres, and the like. The compounds of the invention can also be formulated as prodrugs for ease of delivery. In one exemplary embodiment, the pharmaceutical composition is formulated for oral administration. In other embodiments, the pharmaceutical composition is formulated for parenteral administration.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The compound of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

In therapeutic use for the treatment of pain, the compounds utilized in the pharmaceutical method of the invention are administered at a therapeutically or prophylacticlaly effective dose, e.g., the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound being employed. The dose administered to a patient, in the context of the present invention should be sufficient to effect a beneficial therapeutic response in the patient over time. The size of the dose also will be determined by the existence, nature, and extent of any adverse side-effects that accompany the administration of a particular vector, or transduced cell type in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

In some embodiments, the pharmaceutical composition includes a therapeutically effective amount of a second anti-parasitic compound. Thus, the compounds of the invention can be administered in combination with other therapeutic compounds, either in the same pharmaceutical preparation, or in separate pharmaceutical preparations. The additional therapeutic or prophylactic compounds may be used to treat the same disease as the compound of the invention, e.g., a parasitic disease, a protozoan disease, or a cancer, or can be used to treat a second disease other than the disease treated by the compound of the invention. One or more compounds of the invention can be administered in the same pharmaceutical composition.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

Assays

Anti-parasitic capacities of the compounds of the present invention may be measured using cell culture assays. Cultured mammalian cells that are susceptible to infection by a target protozoan, such as for example, macrophages, erythrocytes, lymphocytes, fibroblasts or other cutaneous cells, hepatocytes, cardiocytes or myocytes are infected with infectious parasitic bodies, such as trypomastigotes to introduce trypanosome infection, merozoites to introduce Plasmodium infection, or promastigotes to introduce Leishmania infection. The culture medium is replaced to remove superfluous infectious parasitic bodies and to add test protease inhibitor compounds. Positive or treated control cultures are given a known parasitic inhibitor. For example, N-methyl piperazine-Phe-homoPhe-vinyl sulfone phenyl (N-Pip-F-hF-VSPh) is known to inhibit trypanosomes. Negative or untreated control cultures are given only diluent (e.g., DMSO) in medium. Cultures are maintained for a time period that encompasses several intracellular cycles of the target parasite in untreated controls, usually about 30 days, but as long as 35, 40, 45 or 50 days or longer, as necessary. Cells are monitored, usually daily but this can be more or less often, for the presence or absence of parasitic infection, usually by contrast phase microscopy. The comparative effectiveness of each test protease inhibitor compound is determined from plots of the duration of the intracellular cycle of the target parasite in treated versus untreated control cultures (generally measured in days).

The ability of compounds of the present invention to inhibit cell invasiveness and migration can also be tested using cellular motility and cellular invasion assays. These assays are particularly applicable to measuring the inhibition of migration of cancer and inflammatory cells. In vitro cellular motility assays are generally carried out using transwell chambers (available from Corning-Costar), with upper and lower culture compartments separated by filters, for example, polycarbonate filters with 8 μm pore size. In vitro cellular invasion assays are conducted using matrigel precoated filters (for example, 100 μg/cm² matrigel on a filter with 8 μm pore size; available from Becton Dickinson). Prior to invasion assays, the matrigel matrix is reconstituted with serum-free cell culture medium. Excess media is removed from the filters and a chemoattractant is placed in the lower compartment of a transwell chamber, for example 5 μg/ml collagen I can be used for a tumor cell. A specified number of cells radiolabeled with ³H-thymidine are seeded onto the filter in motility assays or onto the reconstituted matrigel basement membrane for invasion assays. Cells passing through the filters and attaching to the lower sides of uncoated or matrigel-coated are harvested using trypsin/EDTA, and cell-bound radioactivity is measured in a liquid scintillation counter. The number of migrating cells is calculated by measuring the radioactivity of cells on the underside of a filter in comparison to the radioactivity of a parallel culture containing an identical number of cells to what was originally seeded on the top of the filter or matrigel coating.

The anti-parasitic compounds of the present invention are not limited by any particular mode of action. However, in some embodiments, the anti-parasitic compounds of the present invention are inhibitors of parasitic proteases. The ability of the protease inhibitor compounds to prevent or treat parasitic infections or cancer cell or inflammatory cell invasion or migration in a host subject also can be tested using in vivo disease models. Experimental animal disease models for trypanosomiasis, Leishmania, and malaria are known in the art. For example, murine models for trypanosomiasis are disclosed in Duthie and Kahn, J Immunol (2002) 168:5778, Mucci, et al, Proc Natl Acad Sci (2002) 99:3896, Zuniga, et al, J Immunol (2002) 168:3965 and in Guarner, et al (2001) Am J Trop Med Hyg 65:152. Murine models for Leishmania are described in Rhee, et al, J Exp Med (2002) 195:1565, and in Hommel, et al, Ann Trop Med Parasitol (1995) 89 Supp 1:55. Murine models of malaria are published in Sanni, et al., Methods Mol Med (2002) 72:57, Renia, et al, Methods Mol Med (2002) 72:41, and Li, et al, Med Microbiol Immunol (2001) 189:115. In mouse parasitic disease models, for example, infected mice are administered a test compound of the present invention, and then monitered for amelioration or abatement of infection in comparison to infected, but untreated control mice. Alternatively, uninfected mice are treated with a test compound and then inoculated with a infectious parasitic body to determine the capacity of the compound to prevent parasitic infection. Disease models for cancer and inflammation are also well documented in the published literature. Murine disease models for human cancers require immunodeficient mice (reviewed in Bankert, et al, Front Biosci (2002) 7:c44 and in Hann and Balmain, Curr Opin Cell Biol (2001) 13:778). Additional animal cancer models are discussed in Bast, et al, Cancer Medicine, 5^{th} Ed., B. C. Decker, Hamilton, Ontario, Canada).

The ability of the compounds of the invention may also be screened for effectiveness against proteases (e.g. cathepsin-L like cysteine proteases) in vitro and for effectiveness in disrupting the infectious life cycle of a parasite or malignancy potential of a cancer cell in cell culture and in vivo disease model systems.

For in vitro cysteine protease inhibition determinations, a compound's effectiveness can be given by an IC50 value. In these assays, the enzyme to be inhibited (e.g., a cruzain or cruzipain, a rhodesain, a brucipain, a congopain, a falcipain, CPB2.8 Delta CTE, a cathepsin-L, cathepsin-B, a cathepsin-H, a cathepsin-K, a cathepsin-S) is first incubated with varying concentrations (about 20-50,000 nM) of a test compound. To this is added a short peptide substrate of the enzyme of 1 to 10 amino acids, usually a di- or tri-peptide substrate, which is labeled with either a fluorogenic or chromogenic moiety. An exemplary chromogenic moiety is p-nitro-anilide (pNA). Fluorogenic labels are generally comprised of a fluorescent donor, such as ortho-aminobenzoic acid (Abz) or benzyloxycarbonyl (Z), and a fluorescent quencher, such as 7-(4-methyl)-coumarylamide (AMC), methyl-7-aminocoumarin amide (MCA), 7-amino-4-trifluoromethylcoumarin (AFC) or N-(ethylenediamine)-2,4-dinitrophenyl amide (EDDnp), where the donor and quencher are on either terminus of the peptide substrate. Exemplary peptide substrates include Phe-Arg, Arg-Arg, Phe-Arg-X (X=Ala, Arg), and Phe-X-Ser-Arg-Gln (X=Arg, 4-aminomethyl-phenylalanine (Amf), 4-aminomethyl-N-isopropyl-phenylalanine (Iaf), 4-piperidinyl-alanine (Ppa) or 4-aminocyclohexyl-alanine (Aca)). Cleavage of the labeled substrate induces a chromogenic or fluorescent signal that is measured using spectrophotometer or a spectrofluorometer, respectively. Signals induced in the presence of varying concentrations of test compound are measured in comparison to a positive control of enzyme and substrate and a negative control of enzyme in diluent (e.g., DMSO). Spontaneous cleavage of substrate is measured in controls with substrate alone. IC50 values are determined graphically using compound inhibitor concentrations in the linear portion of a plot of inhibition versus log [I]. Inhibition of a target protease is achieved when the IC50 value is less than about 1000 nM, preferably less than about 500, 300 or 100 nM, more preferably less than about 90, 80, 70, 60, 50, 40, 30, 20 or 10 nM.

Methods

In another aspect, the present invention provides methods of treating or preventing a parasitic disease. The method includes the step of administering to a patient in need thereof a sufficient amount of a pharmaceutical composition of the present invention. As discussed above, the pharmaceutical compositions of the present invention include an anti-parasitic compound of the present invention. Thus, the parasitic disease is treated or prevented by contacting a compound of the present invention with a parasite. In some embodiments, the patient is human.

Diseases caused by a wide variety of parasites may be treated or prevented with the pharmaceutical compositions of the present invention, including those diseases caused by Trypanosoma, Plasmodium, Leishmania, and Trichomonas. More specific exemplary parasites include Trypanosoma cruzi, Trypanosoma brucei gambiense, Trypanosoma brucei rhodesiense, Trypanosoma rangeli, Trypanosoma congolense, Plasmodium falciparum, Plasmodium malariae, Plasmodium vivax, Plasmodium ovale, Leishmania major, Leishmania braziliensis, Leishmania mexicana, Leishmania donvani, Leishmania pifanoi, Leishmania tropica, and Trichomonas Vaginalis.

In one embodiment the parasitic disease is selected from Chagas' disease, African sleeping sickness, nagana, malaria, Leishmaniasis (cutaneous, mucocutaneous or visceral) and STD. In a related embodiment, the STD is trichomoniasis.

Cancer may also be treated or prevented using the methods of the present invention. Methods of treating or preventing cancer include administering to a patient in need thereof a sufficient amount of a pharmaceutical composition including a compound of the present invention.

African sleeping sickness or nagana may be treated or prevented by administering to a patient in need thereof a sufficient amount of a pharmaceutical composition including a therapeutically effective amount of a compound of Formula (I). The compound is contacted with a Trypanosoma brucei parasite thereby treating or preventing African sleeping sickness or nagana.

In an exemplary embodiment, the compound of Formula (I) useful in treating or preventing African sleeping sickness or nagana is an anti-Trypanosoma brucei compound. Exemplary anti-Trypanosoma brucei compounds include the compounds of Formula (I) in which: m is 1; R¹ is attached to the 3′-position or the 4′-position; R¹ is selected from hydrogen, substituted or unsubstituted phenyl, substituted or unsubstituted —NH-phenyl, and substituted or unsubstituted —O-phenyl; R² is methyl; and R³ is selected from —NH₂, substituted or unsubstituted piperidyl, substituted or unsubstituted piperazinyl, —SR^{3A}, and NR^{3B}R^{3C}. R^{3A} and R^{3C} are substituted or unsubstituted (C₁-C₅) alkyl.

In a related embodiment, the anti-Trypanosoma brucei compound includes compounds of Formula (I) in which: m is 1; R¹ is attached to the 3′-position or the 4′-position; R¹ is selected from hydrogen, substituted or unsubstituted phenyl, substituted or unsubstituted —NH-phenyl, and substituted or unsubstituted —O-phenyl; R² is methyl; and R³ is selected from —NH₂, substituted or unsubstituted piperidyl, substituted or unsubstituted piperazinyl, —SR^{3A}, and NR^{3B}R^{3C}. R^{3A} and R^{3C} are substituted or unsubstituted (C₁-C₅) alkyl.

In another exemplary embodiment, the compound of Formula (I) useful in treating or preventing malaria is an anti-Plasmodium falciparum compound. Exemplary anti-Plasmodium falciparum compounds include the compounds of Formula (I) in which: m is 1; X is ═CH—; R¹ is selected from Br and substituted or unsubstituted 4′—NH-phenyl; R² is methyl; and R³ is selected from —N₂ and substituted or unsubstituted piperazinyl. Where R¹ is Br, then R³ is substituted or unsubstituted piperazinyl.

In a related embodiment, the anti-Plasmodium falciparum compound includes compounds of Formula (I) in which: m is 1; X is ═N—; R¹ is selected from hydrogen and -L¹NNHC(S)NH₂; L¹ is selected from substituted or unsubstituted alkylene and substituted or unsubstituted heteroalkylene; R² is methyl; and R³ selected from —NH₂ and —SR^{3A}. R^{3A} is substituted or unsubstituted (C₁-C₅) alkyl.

In yet another exemplary embodiment, the compound of Formula (I) useful in treating or preventing Chagas' Disease is an anti-Trypanosoma cruzi compound. Exemplary anti-Trypanosoma cruzi compounds include the compounds of Formula (I) in which: m is 1; R¹ is selected from hydrogen, substituted or unsubstituted phenyl, substituted or unsubstituted —NH-phenyl, and substituted or unsubstituted —O-phenyl; R¹ is attached to the 3′-position or the 4′-position; R² is methyl; R³ is selected from —NH₂ and substituted or unsubstituted piperazinyl.

In a related embodiment, the anti-Trypanosoma cruzi compound includes compounds of Formula (I) in which: m is 1; X is ═CH—; R¹ is selected from 3′-NH-phenyl, 4′-NH-phenyl, 3′-O-phenyl, 4′-O-phenyl, and 3′-phenyl. In a further related embodiment, R¹ is selected from 3′-NH-phenyl, 4′-NH-phenyl, and 3′-phenyl.

The terms and expressions which have been employed herein are used as terms of description and not of limitation, and there is no intention in the use of such terms and expressions of excluding equivalents of the features shown and described, or portions thereof, it being recognized that various modifications are possible within the scope of the invention claimed. Moreover, any one or more features of any embodiment of the invention may be combined with any one or more other features of any other embodiment of the invention, without departing from the scope of the invention. For example, any feature of the anti-parasitic compounds described above can be incorporated into any of the methods of treating or preventing a parasitic disease without departing from the scope of the invention.

In addition, the patents and scientific references cited herein are incorporated by reference in their entirety for all purposes.

### EXAMPLES

The following examples are provided by way of illustration only and not by way of limitation. Those of skill in the art will readily recognize a variety of noncritical parameters that could be changed or modified to yield essentially similar results.

Protease Inhibition and Parasite Cell Culture Assays

Recombinant cruzain (from T. cruzi) and rhodesain (from T. brucei rhodesiense) were recombinantly expressed as described previously [Eakin et al., Biol. Chem., 268, 6115-8 (1993); Caffrey et al., Mol. Biochem. Parasitol, 118, 61-73 (2001)]. Cruzain (2 nM) or rhodesain (3 nM) was incubated with 0.5 to 10 μM inhibitor in 100 mM sodium acetate, pH 5.5 containing 5 mM DTT (buffer A), for 5 minutes at room temperature. Then buffer A containing Z-Phe-Arg-AMC (Bachem, Km=1 μM) was added to enzyme inhibitor to give 20 gM substrate in 200 μl, and the increase in fluorescence (excitation at 355 nM and emission at 460 nM) was followed with an automated microtiter plate spectrofluorimeter (Molecular Devices, Flex station). Inhibitor stock solutions were prepared at 20 mM in DMSO and serial dilutions were made in DMSO (0.7% DMSO in assay). Controls were performed using enzyme alone, and enzyme with DMSO. IC₅₀ values were determined graphically using inhibitor concentrations in the linear portion of a plot of inhibition versus log [I] (7 concentrations tested with at least 2 in the linear range).

IC₅₀ values against recombinant Falcipain-2 and -3, were determined as described previously [Rosenthal et al., Antimicrob. Agents Chemother, 40, 1600-3 (1996)]. Enzyme was incubated for 30 min at room temperature in 100 mM sodium acetate, pH 5.5, 10 mM DTT with different concentrations of tested inhibitors. Inhibitor solutions were prepared from stock in DMSO (maximum concentration of DMSO in the assay was 1%).

After 30 min. incubation the substrate Z-Leu-Arg-AMC (benzoxycarbonyl-Leu-Arg-7-amino-4-methyl-coumarin) in the same buffer was added to a final concentration of 25 μM. Fluorescence was monitored for 15 min at room temperature in a Fluoroskan Ascent spectrofluorometer (Labsystems). IC₅₀ values were determined from plots of percents of activity over the compound concentration using the data analysis program Prism (GraphPad software).

T. cruzi Culture Assay

Mammalian cells were cultured in RPMI-1640 medium supplemented with 5-10% heatinactivated fetal calf serum (FCS) at 37° C. in 5% CO₂. The Y strain of T. cruzi was maintained by serial passage in bovine embryo skeletal muscle (BESM) cells. Infectious trypomastigotes are collected from culture supernatants. For drug assays, J774 macrophages were irradiated (5000 rad) and plated onto six-well tissue culture plates 24 hr prior to infection with about 106 trypomastigotes/well. Parasites were removed 2 hr postinfection, and the medium was supplemented with the appropriate inhibitor (10 μM). Inhibitor stocks (10 mM) in DMSO were stored at −20° C. J774 monolayers treated with a blank containing DMSO were used as a negative control. RPMI medium with or without inhibitor was replaced every 48 h. Cultures were maintained for up to 46 days and monitored daily by contrast phase microscopy. T. cruzi completed the intracellular cycle in 5-6 days in the untreated controls. The comparative effectiveness of each inhibitor was estimated from plots of the duration of the intracellular cycle of T. cruzi (days) in treated vs untreated control wells.

T. brucei Culture Assay

T. brucei rhodesiense were grown to 10⁶ cells/ml at 37° C. with 5% CO₂ in complete HMI-9 medium containing 10% FBS, 10% Serum Plus, 1× Penicillin/Streptomycin. To carry out drug screens, parasites were diluted to 10⁴ cells/ml in complete HMI-9 medium and aliquoted into 5 ml for growth in culture flasks or 100 ml for growth in 96-well cultures plates. Each inhibitor was added to the appropriate flask or well containing cultured parasites beginning at the highest concentration. The inhibitors were then directly diluted in the cultured parasites by serial dilutions until the concentration of the inhibitors reached 1 nM. Parasites were then incubated in the presence of each inhibitor for 48 hours at 37° C. with 5% CO₂ before monitoring viability. To assay for viability after treatment with inhibitors, parasites were tested for the production of ATP [Rosenthal et al., Antimicrob. Agents Chemother, 40, 1600-3 (1996)]. To do this, 100 ml of parasites from each flask were transferred to 96-well plates. An equal volume of CellTiter-Glo™ (Promega) was added to each well of the transferred parasites or parasites originally grown in 96-well plates separately. The mixture was then shaken at room temperature for 5 minutes before reading the plates using a SpectraFluor Plus multidetection plate reader (Tecan). Alternatively, the treated parasites were counted by hemacytometer 48 hour after incubating them with inhibitors.

P. falciparum Culture Assay

W2-strain P. falciparum parasites (1% parasitemia, 2% hematocrit) were cultured in 0.5 mL of medium in 48-well culture dishes [Trager et al., Science, 193, 673-675 (1976)]. Appropriate inhibitors from 10 mM stocks in DMSO were added to cultured parasites to a final concentration of 20 μM. From 48-well plates, 125 μL of culture was transferred to two 96-well plates (duplicates). Serial dilutions (1:5) of inhibitors were made to final concentrations of 10 μM, 2000 nM, 400 nM, 80 nM, 16 nM, 3.2 nM. Cultures were maintained at 37° C. for 2 days. The parasites were washed and fixed with 1% formaldehyde in PBS. After two days, parasitemia was measured by flow cytometry using the DNA stain YOYO-1 as a marker for cell survival.

Mouse Toxicity Assay

C3H female mice (mean weight, 18 g) (Jackson Laboratories) were injected daily via i.p. with 20 mg/kg weight or 5 mg/kg weight of selected compounds (n=1 per treatment). Compounds were resuspended in 100 μl [70% DMSO (Sigma): 30% ddH20] per dose and injected twice daily for 48 h. Animals were monitored for signs of toxicity, including behavior and feeding, and sacrificed 14 h after the last treatment for necropsy. Major organs were submitted for histological analysis.

Compound Characterization

Proton nuclear magnetic resonance (¹H NMR) spectra were obtained with a Varian Inova-400 MHZ spectrometer with Me₄Si(TMS) as the internal reference. Coupling constants (J) are given in hertz. Elemental and mass spectra analyses were performed at the Micro-Mass facility, University of California, Berkeley. Thin Layer Chromatography (TLC) was carried out on aluminum-backed Merck silica gel 60 F₂₅₄ using the same solvent systems as those used in column chromatography. Column chromatography was performed on silica gel (70-230 mesh). Final products usually precipitated out and were either rinsed with, and/or recrystallised from, methanol. Unless otherwise stated, yields for the reactions were higher than 70%.

1-Biphenyl-2-yl-ethanone thiosemicarbazone (1a): ¹H NMR δ_{H} (400 MHZ; DMSO-d6) 1.77 (s,3H), 7.25 (s, 1H), 7.31 (d, 2H, J=7.6), 7.36 (d, 1H, J=2.0), 7.39 (d, 2H, J=9.2), 7.43 (s, 1H), 7.45 (s, 1H), 7.55 (d, 1H, J=8.4), 8.12 (s, 1H), 10.13 (s, 1H) and 11.95 (s,1H); MS (EI) m/z 270.3 (MH+). Anal. (C₁₅H₁₅N₃S) C, H, N, S.

1-Biphenyl-3-yl-ethanone thiosemicarbazone (1b): ¹H NMR δ_{H} (400 MHz; DMSO-d6) 2.37 (s, 3H), 7.38 (t, 2H, J=7.2), 7.48 (t, 2H, J=8.0), 7.67 (d, 1H, J=7.6), 7.74 (d,2H, J=7.2), 7.91 (d,1H, J=8.0), 8.02 (s, 1H), 8.11 (s, 1H) and 10.23 (s, 1H); MS (EI) m/z 270.3 (MH⁺). Anal. (C₁₅H₁₅N₃S) C, H, N, S.

1-Biphenyl-4-yl-ethanone thiosemicarbazone (1c): ¹H NMR δH (400 MHz; DMSO-d6) 2.34 (s, 3H), 7.39 (t, 1H, J=7.2), 7.49 (t, 2H, J=8.0), 7.69 (dd, 4H, J=7.6), 7.99 (s,1H), 8.03 (d, 2H, J=8.8), 8.30 (s, 1H) and 10.25 (s,1H); MS (EI) m/z 270.3 (MH⁺). Anal. (C₁₅H₁₅N₃S) C, H, N, S.

1-(2-Phenylamino-phenyl)-ethanone thiosemicarbazone (2a): ¹H NMR δH (400 MHz; CDCl₃) 2.25 (s, 1H), 2.35 (s. 3H), 7.05 (m, 6H), 7.28 (m, 4H), 7.41 (d, 1H, J=7.2), and 8.73 (s,1H); MS (EI) m/z 285.3 (MH⁺).

1-(3-Phenylamino-phenyl)-ethanone thiosemicarbazone (2b): δ_{H} (400 MHz; CHCl₃-d1) 2.65 (s, 3H), 6.35 (broad s. 1H), 6.99 (t, 1H, J=7.2), 7.11 (t, 2H, J=8.4), 7.27 (m, 6H), 7.43(s,2H) and 8.73 (s, 1H); MS (EI) m/z 285.3 (MH⁺). Anal. (C₁₅H₁₆N₄S) C, H, N.

1-(4-Phenylamino-phenyl)-ethanone thiosemicarbazone (2c): δ_{H} (400 MHz; CHCl₃-d1) 2.27 (s, 3H), 6.25 (broad s. 2H), 7.04 (d, 2H, J=8.8), 7.15 (d, 1H, J=8.0), 7.26 (s, 2H), 7.32 (t, 2H, J=7.6), 7.63 (d, 2H, J=8.8) and 8.62 (broad s, 1H); MS (EI) m/z 285.3 (MH⁺).

1-(3-Phenoxy-Phenyl)-ethanone thiosemicarbazone (2d): δ_{H} (400 MHz; DMSO-d6) 2.28 (s, 3H), 6.99 (t, 2H, J=8.4), 7.13 (t, 1H, J=6.8), 7.39 (dd, 3H, J=7.2), 7.69 (s, 2H), 7.97 (s, IH) 8.27 (s, 1H) and 10.23 (s, 1H); MS (EI) m/z. 286.2 (MH⁺). Anal. (C₁₅H₁₅N₃OS) C, H, N, S.

1-(4-Phenoxy-Phenyl)-ethanone thiosemicarbazone (2e): δ_{H} (400 MHz; CHCl₃-d1) 2.28 (s, 3H), 6.98 (d, 2H, J=8.4), 7.02 (d, 2H, J=8.4), 7.14 (t, 1H, J=7.6), 7.34 (d, 2H, J=7.6), 7.37 (s, 1H), 7.67 (s, 2H) and 8.82 (s,1H); MS (EI) m/z 286.2 (MH⁺). Anal. (C₁₅H₁₅N₃OS)H, N, S.

3′-Bromoacetophenone thiosemicarbazone (3d): δ_{H} (400 MHz; DMSO-d6) 2.28 (s, 3H), 7.34 (t, 1H, J=8), 7.57 (d, 1H, J=8.4), 7.89 (d, 1H, J=7.6), 8.11 (s, 1H), 8.19 (s, 1H), 8.32 (s, 1H) and 10.25 (s, 1H); MS (EI) m/z 273.2 (MH⁺). Anal. (C₉H₁₀BrN₃S)C, H, N.

1-(3-Amino-phenyl)-ethanone thiosemicarbazone (3e): δ_{H} (400 MHz; DMSO-d6) 2.22 (s, 3H), 5.04 (s, 2H), 6.60 (d, 1H, J=4.4), 7.02 (d, 2H, J=4.4), 7.09 (s, 1H), 7.67 (s, 1H), 8.29 (s,1H), and 10.18 (s,1H); MS (EI) m/z 209.0 (MH⁺). Anal. (C₆H₁₂N₄S)C, H, N, S.

1-(2-Hydroxy-phenyl)-ethanone thiosemicarbazone (3f): δ_{H} (400 MHz; DMSO-d6) 2.31 (s, 3H), 4.49 (s, 1H), 6.84 (s, 1H), 6.86 (t, 2H, J=8.4), 7.25 (t, 1H, J=7.6), 7.53 (s, 2H); MS (EI) m/z 210.2 (MH⁺).

1-(3-Hydroxy-phenyl)-ethanone thiosemicarbazone (3g): δ_{H} (400 MHz; DMSO-d6) 2.25 (s, 3H), 6.79 (d, 1H, J=8.0), 7.19 (t, 1H, J=8.0), 7.24 (s, 1H), 7.33 (d, 1H, J=7.6), 7.78 (s, 1H), 8.27 (s, 1H) 9.43 (s, 1H) and 10.21 (s, 1H); MS (EI) m/z 210.2 (MH⁺). Anal. (C₉H₁₁N₃OS)C, H, N, S.

1-(4-Hydroxy-phenyl)-ethanone thiosemicarbazone (3h): δ_{H} (400; DMSO-d6) 2.23 (s, 3H), 6.75 (d, 2H, J=8.8), 7.76 (d, 2H, J=8.4), 7.80 (s, 1H), 8.16 (s, 1H), 9.72 (s,1H), and 10.06 (s,1H); MS (EI) m/z. 210.2 (MH⁺). Anal. (C₆H₁₁N₃OS)C, H, N, S.

Methyl 3-[1-(3′-bromophenyl)ethylidene]hydrazinecarbodithioate (4a): δ_{H} (400 MHz; DMSO-d6) 2.38 (s, 3H), 2.52 (s, 3H), 7.42 (t, 1H, J=8), 7.65 (d, 1H, J=7.6), 7.85 (d, 1H, J=8.0), 8.02 (s, 1H) and 12.53 (s,1H); MS (EI) m/z 304.3 (MH⁺). Anal. (C₁₀H₁₁BrN₂S₂) C, H, N, S.

Piperidine-1-carbothioic acid [1-(3-bromo-phenyl)-ethylidene]-hydrazide (4c): δ_{H} (400 M; DMSO-d6) 1.61 (s,6H), 2.27 (s, 3H), 3.83 (s,4H), 7.38 (t, 1H, J=7.6), 7.58 (d, 1H, J=7.6), 7.55 (d, 1H, J=7.6), 7.93 (s, 1H) and 9.66 (s,1H); MS (EI) m/z 340.3 (MH⁺). Anal. (C₁₄H₁₈BrN₃S)C, H, N.

4-Methyl-piperazine-1-carbothioic acid [1-(3-bromo-phenyl)-ethylidene]-hydrazide (4d): δ_{H} (400 z; DMSO-d6) 2.21 (s,3H), 2.28 (s, 3H, 2.39 (s,4H), 3.86 (s,4H), 7.39 (t, 1H, J=7.2), 7.59 (d, 1H, J=6.8), 7.55 (d, 1H, J=7.6), 7.94 (s, 1H) and 9.86 (s,1H); MS (EI) m/z 355.5 (MH⁺). Anal. (C₁₄H₁₉BrN₄S)C, H, N.

3′-Bromoacetophenone 4,4-diethyl-3-thiosemicarbazone (4e): δ_{H} (400 MHz; DMSOd6) 1.20 (t,6H), 2.28 (s, 3H), 3.73 (q, 4H), 7.39 (t, 1H, J=8.0), 7.59 (d, 1H, J=7.6), 7.76 (d, 1H, J=8.0), 7.95 (s, 1H) and 9.45 (s, 1H); MS (EI) m/z 328.3 (MH⁺)

1-Pyridin-2-yl-ethanon e thiosemicarbazone (3a): δ_{H} (400 MHz; DMSO-d6) 2.39 (s, 3H), 7.38 (dt, 1H, J=1.2), 7.79 (dt, 1H, J=2.4), 8.13 (s, 1H), 8.42 (t, 2H, J=8.0), 8.57 (d,1H, J=4.4) and 10.31 (s, 1H); MS (EI) m/z 195.3 (MH⁺). Anal. (C₈H₁₀N₄S.0.5H₂O)C, H, N, S.

1-Pyridin-3-yl-ethanone thiosemicarbazone (3b): δ_{H} (400; DMSO-d6) 2.28 (s, 3H), 7.35 (dd, 1H, J=4.4), 8.04 (s, 1H), 8.29 (d, 2H, J=6.8), 8.51 (d, 1H, J=3.6), 9.06 (s,1H) and 10.28 (s,1H); MS (EI) m/z 195.3 (MH⁺). Anal. (C₈H₁₀N₄S)C, H, N, S.

1-Pyridin-4-yl-ethanone thiosemicarbazone (3c): δ_{H} (400; DMSO-d6) 2.30 (s, 3H), 7.90 (dd, 2H; J=2.0), 8.14 (s, 1H), 8.44 (s, 1H), 8.58 (dd, 2H, J=1.6) and 10.42 (s,1H); MS (EI) m/z 195.3 (MH⁺). Anal. (C₆H₁₀N₄S)C, H, N, S.

N′-(1-Pyridin-2-yl-ethylidene)-hydrazinecarbodithioic acid methyl ester (4b): δ_{H} (400 MHz; DMSO-d6) 2.26 (s, 3H), 2.50 (s, 3H), 7.44 (t, 1H, J=8.4), 7.67 (d, 1H, J=8.0), 7.90 (d, 1H, J=8.0), 8.65 (s, 1H); MS (E1) m/z 226.3 (MH⁺). Anal. (C₆H₁₁N₃S₂) C, H, N, S.

1-(3-Acetyl-phenyl)-ethanone thiosemicarbazone (3i): δ_{H} (400 MHz; DMSO-d6) 2.35 (t, 6H), 7.40 (t, 1H, J=7.6), 7.97 (dd, 4H, J=1.6), 8.19 (s, 1H), 8.28(s, 2H) and 10.22 (s, 2H); MS (E1) m/z 309.4 (MH+). Anal. (C₁₂H₁₆N₆S₂.H₂0) H, N, S.

1-(6-Acetyl-pyridin-2-yl)-ethanone thiosemicarbazone (3j): δ_{H} (400 MHz; DMSO-d6) 2.44 (s, 6H), 7.78 (t, 1H, J=7.6), 8.16 (broad s, 2H), 8.43 (d, 4H, J=8.4), and 10.31 (s, 2H); MS (EI) m/z 310.3 (MH⁺). Anal. (C₁₁H₁₅N₇S₂.1.5H₂O)C, H.

1-(5-Acetyl-2,6-dimethyl-pyridin-3-yl)-ethanone thiosemicarbazone(3k): δ_{H} (400 MHz; DMSO-d6) 2.21 (s, 6H), 2.29 (t,6H, J=6.8), 7.68 (d, 1H, J=2.8), 8.24 (broad s, 4H), and 10.23 (s, 2H); MS (EI) m/z 338.1 (MH⁺). Anal. (C₁₃H₁₉N₇S₂) H, N, S

What is claimed is:

1. A compound having the formula: 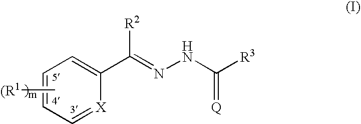
wherein
Q is a member selected from ═S and ═O;
m is an integer from 1 to 3;
X is a member selected from ═CH— and ═N—;
R¹ is a member independently selected from hydrogen, Br, unsubstituted alkyl, substituted or unsubstituted heterocycloalkyl, substituted or unsubstituted cycloalkyl, substituted or unsubstituted aryl, substituted or unsubstituted heteroaryl, —OR^{1A}, and —NR^{1B}R^{1C}, wherein
R^{1A} is a member selected from (C₃-C₁₀) unsubstituted alkyl, substituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl;
R^{1B} and R^{1C} are members independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl;
R² is substituted or unsubstituted alkyl;
R³ is a member selected from substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —SR^{3A}, and —NR^{3B}R^{3C}, wherein
R^{3A} is a member selected from substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl, and
R^{3B} and R^{3C} are members independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl, wherein
R^{3B} and R^{3C} are optionally joined together to form a substituted or unsubstituted ring with the nitrogen to which they are attached;
wherein if m is 1 and R¹ is Br, then at least one of R^{3B} and R^{3C} is selected from substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl; and
wherein if R¹ is hydrogen and Q is ═S, then R³ is not —NR^{3B}R^{3C}.

2. The compound of claim 1, wherein
m is 1;
R¹ is a member selected from hydrogen, Br, substituted or unsubstituted aryl, —OR^{1A}, —NR^{1B}R^{1C}, and -L¹NNHC(S)NH₂, wherein
R^{1A} is substituted or unsubstituted aryl,
R^{1B} is hydrogen,
R^{1C} is substituted or unsubstituted aryl, and
L¹ is a member selected from substituted or unsubstituted alkylene and substituted or unsubstituted heteroalkylene;
R² is methyl; and
R³ is a member selected from —SR^{3A}, and —NR^{3B}R^{3C}, wherein
R^{3A} is a member selected from substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, and
R^{3B} and R^{3C} are members independently selected from hydrogen, substituted or unsubstituted alkyl, wherein
R^{3B} and R^{3C} are optionally joined together to form a substituted or unsubstituted ring with the nitrogen to which they are attached, wherein said ring is a member selected from substituted or unsubstituted piperidyl, substituted or unsubstituted piperizyl, and substituted or unsubstituted pyridyl.

3. The compound of claim 1, wherein
m is 1;
R¹ is a member selected from hydrogen, substituted or unsubstituted phenyl, substituted or unsubstituted —NH-phenyl, and substituted or unsubstituted —O-phenyl, wherein
R¹ is attached to the 3′-position or the 4′-position;
R² is methyl; and
R³ is a member selected from —NH₂, substituted or unsubstituted piperidyl, substituted or unsubstituted piperazinyl, —SR^{3A}, and NR^{3B}R^{3C}, wherein
R^{3A} and R^{3C} are substituted or unsubstituted (C₁-C₅) alkyl.

4. The compound of claim 3, wherein
R³ is a member selected from substituted or unsubstituted piperidyl, and substituted or unsubstituted piperazinyl, wherein R^{3A} and R^{3C} are substituted or unsubstituted (C₁-C₅) alkyl.

5. The compound of claim 3, wherein
X is ═N—;
R¹ is hydrogen; and
R³ is —SR^{3A}.

6. The compound of claim 1, wherein
m is 1;
R¹ is a member selected from hydrogen, substituted or unsubstituted phenyl, substituted or unsubstituted —NH-phenyl, and substituted or unsubstituted —O-phenyl, wherein R¹ is attached to the 3′-position or the 4′-position;
R² is methyl; and
R³ is a member selected from —NH₂ and substituted or unsubstituted piperazinyl.

7. The compound of claim 6, wherein
X is ═CH—; and
R¹ is a member selected from 3′-NH-phenyl, 4′-NH-phenyl, 3′-O-phenyl, 4′-O-phenyl, and 3′-phenyl.

8. The compound of claim 7, wherein R¹ is a member selected from 3′-NH-phenyl, 4′-NH-phenyl, and 3′-phenyl.

9. The compound of claim 1, wherein
m is 1;
X is ═CH—
R¹ is a member selected from Br and substituted or unsubstituted 4′-NH-phenyl;
R² is methyl;
R³ is a member selected from —NH₂ and substituted or unsubstituted piperazinyl; and
wherein if R¹ is Br, then R³ is substituted or unsubstituted piperazinyl.

10. The compound of claim 1, wherein
m is 1;
X is ═N—;
R¹ is a member selected from hydrogen and -L¹NNHC(S)NH₂, wherein L¹ is a member selected from substituted or unsubstituted alkylene and substituted or unsubstituted heteroalkylene;
R² is methyl; and
R³ a member selected from —NH₂ and —SR^{3A}, wherein R^{3A} is substituted or unsubstituted (C₁-C₅) alkyl.

11. A compound having the formula: 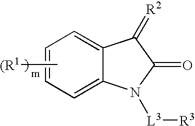
wherein
m is an integer from 1 to 3;
R¹ is a member selected from hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl;
R² is a member selected from ═O and ═N—NH—C(Q)-NR^{2A}R^{2B}, wherein
Q is a member selected from ═S and ═O;
R^{2A} and R^{2B} are members independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl;
L³ is a member selected from substituted or unsubstituted alkylene, substituted or unsubstituted heteroalkylene, substituted or unsubstituted cycloalkylene, substituted or unsubstituted heterocycloalkylene, substituted or unsubstituted arylene, and substituted or unsubstituted heteroarylene;
R³ is a member selected from substituted or unsubstituted heterocycloalkyl, substituted or unsubstituted quinolinyl, and NR^{3A}R^{3B}, wherein
R^{3A} and R^{3B} are members independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

12. The compound of claim 11, wherein
m is an integer from 1 to 2;
R¹ is a member selected from hydrogen, halogen, and unsubstituted alkyl;
Q is ═S;
R^{2A} and R^{2B} are hydrogen;
L³ is a member selected from unsubstituted alkylene, unsubstituted heteroalkylene, and unsubstituted heterocycloalkylene; and
R³ is a member selected from unsubstituted quinolinyl, quinolinyl substituted with a halogen, substituted or unsubstituted piperidinyl, substituted or unsubstituted morpholinyl, substituted or unsubstituted piperazinyl, and NR^{3A}R^{3B}, wherein R^{3A} and R^{3B} are unsubstituted alkyl.

13. A compound having the formula: 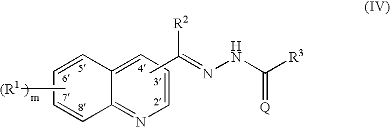
Q is a member selected from ═S and ═O;
m is an integer from 1 to 6;
R¹ is a member independently selected from hydrogen, halogen, substituted or unsubstituted alkyl, substituted or unsubstituted heterocycloalkyl, substituted or unsubstituted cycloalkyl, substituted or unsubstituted aryl, substituted or unsubstituted heteroaryl, —OR^{1A}, and —NR^{1B}R^{1C}, wherein
R^{1A} is a member selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl;
R^{1B} and R^{1C} are members independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl;
R² is substituted or unsubstituted alkyl;
R³ is a member selected from substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —SR^{3A}, and —NR^{3B}R^{3C}, wherein
R^{3A} is a member selected from substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl, and
R^{3B} and R^{3C} are members independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl, wherein
R^{3B} and R^{3C} are optionally joined together to form a substituted or unsubstituted ring with the nitrogen to which they are attached; and
wherein if R¹ is hydrogen and Q is ═S, then only hydrogen or R¹ is attached to the 2′ position.

14. A compound having the formula 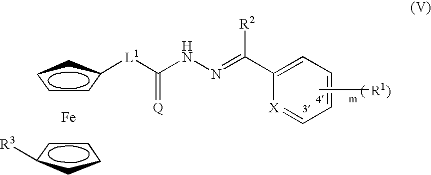
wherein
Q is a member selected from ═S and ═O;
m is an integer from 1 to 3;
X is a member selected from ═CH— and ═N—;
L¹ is a member selected from substituted or unsubstituted alkylene, substituted or unsubstituted heteroalkylene, substituted or unsubstituted cycloalkylene, substituted or unsubstituted heterocycloalkylene, substituted or unsubstituted arylene, and substituted or unsubstituted heteroarylene;
R¹ is a member independently selected from hydrogen, halogen, substituted or unsubstituted alkyl, substituted or unsubstituted heterocycloalkyl, substituted or unsubstituted cycloalkyl, substituted or unsubstituted aryl, substituted or unsubstituted heteroaryl, —OR^{1A}, and —NR^{1B}R^{1C}, wherein
R^{1A} is a member selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl;
R^{1B} and R^{1C} are members independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl;
R² is substituted or unsubstituted alkyl;
R³ is a member selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

15. A compound having the formula 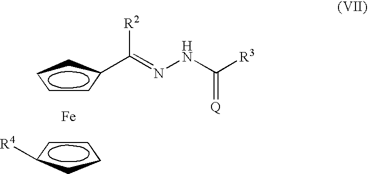
Q is a member selected from ═S and ═O;
R² is substituted or unsubstituted alkyl;
R³ is a member selected from substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —SR^{3A}, and —NR^{3B}R^{3C}, wherein
R^{3A} is a member selected from substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl, and
R^{3B} and R^{3C} are members independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl, wherein
R^{3B} and R^{3C} are optionally joined together to form a substituted or unsubstituted ring with the nitrogen to which they are attached.
R⁴ is a member selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
wherein if R⁴ is hydrogen, then R³ is not —NR^{3B}R^{3C}.

16. A pharmaceutical composition comprising a therapeutically effective amount of a compound of claim 1 and a physiologically acceptable carrier.

17. The pharmaceutical composition of claim 16, the composition comprising a therapeutically effective amount of a second anti-parasitic compound.

18. The pharmaceutical composition of claim 16, wherein the composition is formulated for oral administration.

19. The pharmaceutical composition of claim 16, wherein the composition is formulated for parenteral administration.

20. A method of treating a parasitic disease, said method comprising the step of administering to a patient in need thereof a sufficient amount of a pharmaceutical composition according to claim 16, wherein said compound is contacted with a parasite thereby treating said parasitic disease.

21. The method of claim 20, wherein said parasite is selected from the group consisting of Trypanosoma, Plasmodium, Leishmania, and Trichomonas and said parasitic disease is selected from the group consisting of Chagas' disease, African sleeping sickness, nagana, malaria, Leishmaniasis, and STD.

22. The method of claim 21, wherein said parasite is Trichomonas and said parasitic disease is STD.

23. The method of claim 21, wherein said parasite is Plasmodium and said parasitic disease is malaria.

24. The method of claim 21, wherein said parasite is Leishmania and said parasitic disease is Leishmaniasis.

25. The method of claim 21, wherein said parasite is Trypanosoma and said parasitic disease is Chagas' disease, African sleeping sickness, or nagana.

26. The method of claim 21, wherein the patient is a human.

27. A method of preventing a parasitic infection, said method comprising the step of administering to a patient in need thereof a sufficient amount of a pharmaceutical composition according to claim 16, wherein said compound is contacted with a parasite thereby preventing said parasitic disease.

28. The method of claim 27, wherein said parasite is selected from the group consisting of Trypanosoma, Plasmodium, Leishmania, and Trichomonas, and said parasitic disease is selected from the group consisting of Chagas' disease, African sleeping sickness, nagana, malaria, Leishmaniasis, and trichomoniasis.

29. The method of claim 28, wherein said parasite is Trichomonas and said parasitic disease is STD.

30. The method of claim 28, wherein said parasite is Plasmodium and said parasitic disease is malaria.

31. The method of claim 28, wherein said parasite is Leishmania and said parasitic disease is Leishmaniasis.

32. The method of claim 28, wherein said parasite is Trypanosoma and said parasitic disease is Chagas' disease, African sleeping sickness, or nagana.

33. The method of claim 28, wherein the patient is a human.

34. A method of treating or preventing cancer, said method comprising the step of:
administering to a patient in need thereof a sufficient amount of a pharmaceutical composition comprising a compound of claim 16.

35. A method of treating or preventing African sleeping sickness or nagana, said method comprising the step of administering to a patient in need thereof a sufficient amount of a pharmaceutical composition comprising a therapeutically effective amount of a compound of claim 3, wherein said compound is contacted with a Trypanosoma brucei parasite thereby treating or preventing African sleeping sickness or nagana.

36. A method of treating or preventing malaria, said method comprising the step of administering to a patient in need thereof a sufficient amount of a pharmaceutical composition comprising a therapeutically effective amount of a compound of claim 9, wherein said compound is contacted with a Plasmodium falciparum parasite thereby treating or preventing malaria.

37. A method of treating or preventing malaria, said method comprising the step of administering to a patient in need thereof a sufficient amount of a pharmaceutical composition comprising a therapeutically effective amount of a compound of claim 10, wherein said compound is contacted with a Plasmodium falciparum parasite thereby treating or preventing malaria.

38. A method of treating or preventing Chagas' Disease, said method comprising the step of administering to a patient in need thereof a sufficient amount of a pharmaceutical composition comprising a therapeutically effective amount of a compound of claim 6, wherein said compound is contacted with a Trypanosoma cruzi parasite thereby treating or preventing Chagas' Disease.
